(19) United States
(12) Patent Application Publication
(10) Pub. No.: US 2024/0002400 A1
(43) Pub. Date: Jan. 4, 2024

(54) ENANTIOSELECTIVE ALKENYLATION OF ALDEHYDES

(71) Applicant: AMGEN INC., THOUSAND OAKS, CA (US)

(72) Inventors: KYLE D. BAUCOM, VENTURA, CA (US); MICHAEL T. CORBETT, SAN CARLOS, CA (US); SHENG CUI, LEXINGTON, MA (US); NEIL F. LANGILLE, SUDBURY, MA (US); ANDREAS RENE ROTHELI, WELLESLEY, MA (US); ROBERTO PROFETA, OCCHIOBELLO, ROVIGO (IT); AUSTIN G. SMITH, CARY, NC (US)

(73) Assignee: AMGEN INC., THOUSAND OAKS, CA (US)

(21) Appl. No.: 18/038,579

(22) PCT Filed: Nov. 23, 2021
(86) PCT No.: PCT/US2021/060447

§ 371 Date: May 24, 2023

Related U.S. Application Data
(60) Provisional application No. 63/118,057, filed on Nov. 25, 2020.

(51) Int. Cl. C07D 513/00; C07D 519/00

(52) U.S. Cl. CPC C07D 513/00; C07D 519/00

(57) ABSTRACT

Provided herein are processes for synthesizing intermediates useful in preparing Mcl-1 inhibitors. In particular, provided herein are processes for synthesizing compound IA, wherein R¹ is described herein. Compound IA can be useful in synthesizing compound A1, or a salt or solvate thereof, and compound A2, or a salt of solvate thereof.

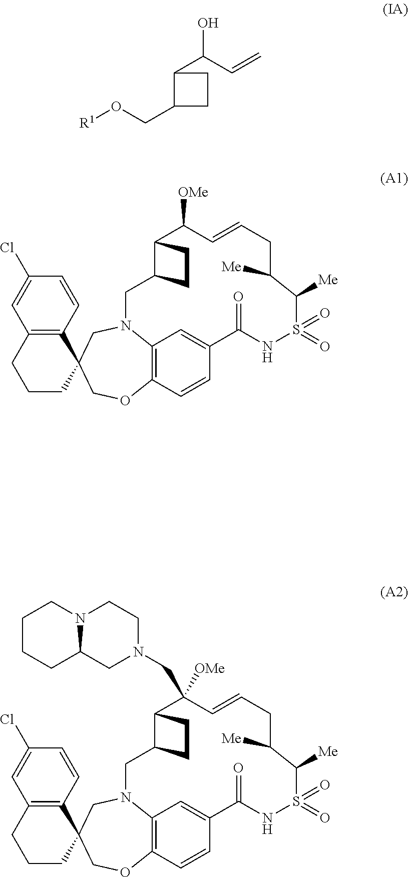

### CROSS REFERENCES TO RELATED APPLICATIONS

This application claims the benefit of U.S. Provisional Application No. 63/118,057, filed on Nov. 25, 2020, which is hereby incorporated by reference in its entirety and for all purposes as if fully set forth herein.

### FIELD OF THE INVENTION

The present disclosure relates to processes for synthesizing and purifying intermediates useful in preparing (1S,3′R,6′R,7'S,8′E,11'S,12′R)-6-chloro-7′-methoxy-11′,12′-dimethyl-3,4-dihydro-2H,15′H-spiro[naphthalene-1,22′[20]oxa[13]thia[1,14]diazatetracyclo[14.7.2.0^{3,6}0.0^{19,24}]pentacosa[8,16,18,24]tetraen]-15′-one 13′,13′-dioxide (compound A1; AMG 176), a salt, or solvate thereof, and in preparing (1S,3′R,6′R,7′R,8′E,11′S,12′R)-6-chloro-7′-methoxy-11′,12′-dinethyl-7′-((9aR)-octahydro-2H-pyrido[1,2-a]pyrazin-2-ylmethyl)-3,4-dihydro-2H,15′H-spiro[naphthalene-1,22′-[20]oxa[13]thia[1,14]diazatetracyclo[14.7.2.0^{3,6}.0^{19,24}]pentacosa [8,16,18,24]tetraen]-15′-one 13′,13′-dioxide (compound A2; AMG 397), a salt, or solvate thereof. These compounds are inhibitors of myeloid cell leukemia 1 protein (Mcl-1).

### BACKGROUND OF THE INVENTION

The compound, (1S,3′R,6′R,7′S,8′E,11′S,12′R)-6-chloro-7′-methoxy-11′,12′-dimethyl-3,4-dihydro-2H,15′H-spiro[naphthalene-1,22′[20]oxa[13]thia[1,14]diazatetracyclo[14.7.2.0^{3,6}0.0^{19,24}]pentacosa[8,16,18,24]tetraen]-15′-one 13′,13′-dioxide (compound A1), is useful as an inhibitor of myeloid cell leukemia 1 (Mcl-1):

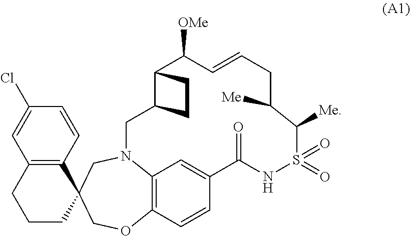

The compound, (1S,3′R,6′R,7′R,8′E,11′S,12′R)-6-chloro-7′-methoxy-11′,12′-dinethyl-7′-((9aR)-octahydro-2H-pyrido[1,2-a]pyrazin-2-ylmethyl)-3,4-dihydro-2H,15′H-spiro[naphthalene-1,22′-[20]oxa[13]thia[1,14]diazatetracyclo [14.7.2.0^{3,6}.0^{19,24}]pentacosa[8,16,18,24]tetraen]-15′-one 13′,13′-dioxide (compound A2), is useful as an inhibitor of myeloid cell leukemia 1 (Mcl-1):

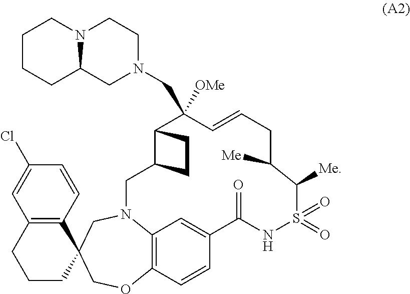

One common characteristic of human cancer is overexpression of MCI-1. Mcl-1 overexpression prevents cancer cells from undergoing programmed cell death (apoptosis), allowing the cells to survive despite widespread genetic damage.

Mcl-1 is a member of the Bcl-2 family of proteins. The Bcl-2 family includes pro-apoptotic members (such as BAX and BAK) which, upon activation, form a homo-oligomer in the outer mitochondrial membrane that leads to pore formation and the escape of mitochondrial contents, a step in triggering apoptosis. Antiapoptotic members of the Bcl-2 family (such as Bcl-2, Bcl-XL, and Mcl-1) block the activity of BAX and BAK. Other proteins (such as BID, BIM, BIK, and BAD) exhibit additional regulatory functions. Research has shown that Mcl-1 inhibitors can be useful for the treatment of cancers. Mcl-1 is overexpressed in numerous cancers.

U.S. Pat. No. 9,562,061, which is incorporated herein by reference in its entirety, discloses compound A1 as an Mcl-1 inhibitor and provides a method for preparing it. However, improved synthetic methods that result in greater yield and purity of compound A1 are desired, particularly for the commercial production of compound A1.

U.S. Pat. No. 10,300,075, which is incorporated herein by reference in its entirety, discloses compound A2 as an Mcl-1 inhibitor and provides a method for preparing it. However, improved synthetic methods that result in greater yield and purity of compound A2 are desired, particularly for the commercial production of compound A2.

### SUMMARY OF THE INVENTION

In one aspect, the invention provides a method of synthesizing a compound of Formula BI, the compound of Formula BI having the formula

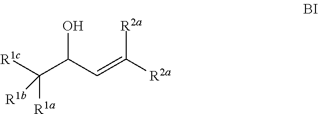

wherein the method comprises:
a) reacting a compound of Formula BII with an alkenyl boron compound and a catalyst in the presence of a base and optionally a solvent to form a product mixture comprising the compound of Formula BI, wherein the catalyst is prepared from a copper I salt or a copper II salt and a phosphine;
wherein the phosphine is at least two equivalents of a monophosphine or at least one equivalent of a diphosphine with respect to the copper I salt or is at least four equivalents of a monophosphine or at least two equivalents of a disphosphine with respect to the copper II salt;
and further wherein the sp² hybridized carbon atom of the alkenyl group that is not directly bonded to the boron atom of the alkenyl boron compound is bonded to 2 R^{2a} groups, wherein each R^{2a} is independently selected from —H, —C₁-C₆ alkyl, or a —C₆-C₁₀ aryl group, wherein the aryl group is unsubstituted or is substituted with 1 or 2 R^{3a} groups independently selected from —C₁-C₆ alkyl, —NO₂, halo, or —O—C₁-C₆; and further wherein if one of the R^{2a} groups is an aryl group, then the other R^{2a} group is not an aryl group; wherein, BII has the formula

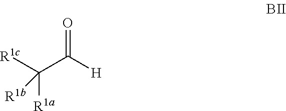

wherein
R^{1a}R^{1b} and R^{1c} are independently selected from —H, or —C₁-C₆ alkyl, or OR^{1d} wherein R^{1d} is selected from —H, —C₁-C₆ alkyl, —Si(C₁-C₆ alkyl)₃, or C₁-C₆ alkyl-aryl, wherein the aryl in an Rid group is a C₆-C₁₀ aromatic ring that is unsubstituted, or is substituted with 1, 2, or 3 substituents selected from —OH, —O—C₁-C₆ alkyl, —O—Si(C₁-C₆ alkyl)₃, halo, or C₁-C₆ haloalkyl;
or R^{1a} and R^{1b} may join to form a ring comprising 3, 4, 5, 6, 7, or 8 ring members comprising 0 or 1 oxygen atom, wherein the ring is unsubstituted or is substituted with 1 or 2 substituents selected from —OR^{1e} or —C₁-C₆—OR^{1e};
R^{1e} is selected from —H, —C₁-C₆ alkyl, —CH₂-aryl, —Si(C₁-C₆ alkyl)₃, tetrahydropyranyl, aryl, or —C═O—C₁-C₆ alkyl, wherein the aryl of an R^{1e} group is a C₆-C₁₀ aromatic ring that is unsubstituted or is substituted with 1, 2, or 3 substituents selected from —OR^{1f}, -halo, —C₁-C₆ alkyl, —C₁-C₆ haloalkyl, or —C═O—C₁-C₆ alkyl;
R^{1f} is selected from —H, —C₁-C₆ alkyl, —Si(C₁-C₆ alkyl)₃, tetrahydropyranyl, or —(C₁-C₆ alkyl)-aryl, wherein the aryl of an R^{1f} group is a C₆-C₁₀ aromatic ring that is unsubstituted, or is substituted with 1, 2, or 3 substituents selected from —OH, —O—C₁-C₆ alkyl, —O—Si(C₁-C₆ alkyl)₃, halo, or C₁-C₆ haloalkyl; and
b) reacting the product mixture with an oxidizing agent to oxidize the phosphine moieties in the phosphine to phosphine oxides to produce an oxidized phosphine.

In another aspect, the invention provides a method of synthesizing a compound of Formula IA′, the compound of Formula IA′ having the formula:

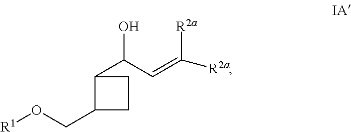

wherein the method comprises:
reacting a compound of Formula IIA with an alkenyl boron compound and a catalyst in the presence of a base and an optional solvent to form a product mixture comprising the compound of Formula IA′, wherein the catalyst is prepared from a copper I salt or a copper II salt and a phosphine, wherein the phosphine is at least two equivalents of a monophosphine or at least one equivalent of a diphosphine with respect to the copper I salt or is at least four equivalents of a monophosphine or at least two equivalents of a disphosphine with respect to the copper II salt,
and further wherein the sp² hybridized carbon atom of the alkenyl group that is not directly bonded to the boron atom of the alkenyl boron compound is bonded to 2 R^{2a} groups, wherein each R^{2a} is independently selected from —H, —C₁-C₆ alkyl, or a —C₆-C₁₀ aryl group, wherein the aryl group is unsubstituted or is substituted with 1 or 2 R^{3a} groups independently selected from —C₁-C₆ alkyl, —NO₂, halo, or —O—C₁-C₆ alkyl; and further wherein if one of the R^{2a} groups is an aryl group, then the other R^{2a} group is not an aryl group;
wherein the compound of Formula IIA has the structure

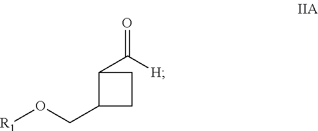

wherein R¹ is selected from —H, —C₁-C₆ alkyl, —C═O—C₁-C₆ alkyl, —C═O-aryl, —Si(C₁-C₆ alkyl)₃, tetrahydropyranyl, or —C₁-C₆ alkyl-aryl, wherein the aryl group in an R¹ group is a C₆-C₁₀ aromatic ring that is unsubstituted, or is substituted with 1, 2, or 3 substituents selected from —OH, NO₂, —O—C₁-C₆ alkyl, halo, or C₁-C₆ haloalkyl.

In another aspect, the invention provides a compound of Formula IA having the formula

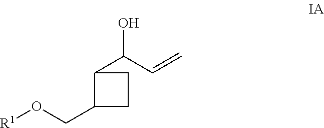

wherein R¹ is selected from —H, —C₁-C₆ alkyl, C═O—C₁-C₆ alkyl, —Si(C₁-C₆ alkyl)₃, tetrahydropyranyl, —C₁-C₆ alkyl-aryl, wherein the aryl in an R¹ group is a C₆-C₁₀ aromatic ring that is unsubstituted, or is substituted with 1, 2, or 3 substituents selected from —OH, —O—C₁-C₆ alkyl, halo, or C₁-C₆ haloalkyl.

In yet another aspect, the invention provides a method for synthesizing compound A3 using compound IA, wherein the compound A3 has the following structure:

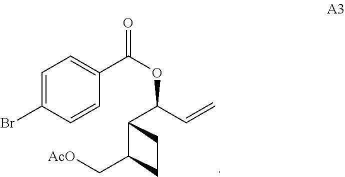 [SMILES: C=C[C@H](OC(=O)c1ccc(Br)cc1)[C@@H]1CC[C@H]1COC(C)=O]

In yet another aspect, the invention provides a method for synthesizing compound A1 using compound IA, wherein the compound A1 has the following structure:

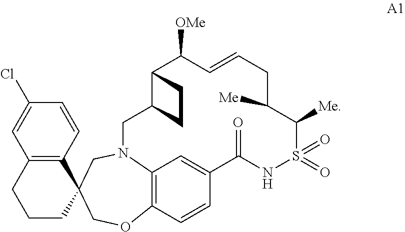

In yet another aspect, the invention provides a method for synthesizing compound A2 using compound IA, wherein the compound A2 has the following structure:

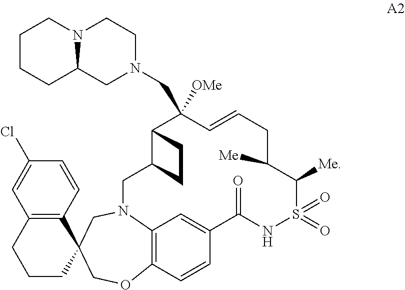

Other objects, features and advantages of the invention will become apparent to those skilled in the art from the following description and claims.

### DETAILED DESCRIPTION OF THE INVENTION

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound. The compounds of the present disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

The term “comprising” is meant to be open ended, i.e., all encompassing and non-limiting. It may be used herein synonymously with “having” or “including”. Comprising is intended to include each and every indicated or recited component or element(s) while not excluding any other components or elements. For example, if a composition is said to comprise A and B. This means that the composition has A and B in it, but may also include C or even C, D, E, and other additional components.

Certain compounds of the invention may possess asymmetric carbon atoms (optical centers) or double bonds; the racemates, enantiomers, diastereomers, geometric isomers and individual isomers are all intended to be encompassed within the scope of the invention. Furthermore, atropisomers and mixtures thereof such as those resulting from restricted rotation about two aromatic or heteroaromatic rings bonded to one another are intended to be encompassed within the scope of the invention. For example, when R⁴ is a phenyl group and is substituted with two groups bonded to the C atoms adjacent to the point of attachment to the N atom of the pyrimidinone, then rotation of the phenyl may be restricted. In some instances, the barrier of rotation is high enough that the different atropisomers may be separated and isolated.

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the mirror image enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. If the stereochemistry of a structure or a portion of a structure is not indicated with, for example, bold or dashed lines, the structure or portion of the structure is to be interpreted as encompassing all stereoisomers of it. A bond drawn with a wavy line indicates that both stereoisomers are encompassed. This is not to be confused with a wavy line drawn perpendicular to a bond which indicates the point of attachment of a group to the rest of the molecule.

As described above, this invention encompasses the use of stereomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al. (1997) Tetrahedron 33:2725; Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN, 1972).

As known by those skilled in the art, certain compounds of the invention may exist in one or more tautomeric forms. Because one chemical structure may only be used to represent one tautomeric form, it will be understood that for convenience, referral to a compound of a given structural formula includes tautomers of the structure represented by the structural formula.

The term “solvate” refers to the compound formed by the interaction of a solvent and a compound. Suitable solvates are pharmaceutically acceptable solvates, such as hydrates, including monohydrates and hemi-hydrates.

The compounds of the invention may also contain naturally occurring or unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabeled compounds are useful as therapeutic or prophylactic agents, research reagents, e.g., assay reagents, and diagnostic agents, e.g., in vivo imaging agents. All isotopic variations of the compounds of the invention, whether radioactive or not, are intended to be encompassed within the scope of the invention. For example, if a variable is said or shown to be H, this means that variable may also be deuterium (D) or tritium (T).

“Alkyl” refers to a saturated branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl and propan-2-yl, butyls such as butan-1-yl, butan-2-yl, 2-methyl-propan-1-yl, 2-methyl-propan-2-yl, tert-butyl, and the like. In certain embodiments, an alkyl group comprises 1 to 20 carbon atoms. In some embodiments, alkyl groups include 1 to 10 carbon atoms or 1 to 6 carbon atoms whereas in other embodiments, alkyl groups include 1 to 4 carbon atoms. In still other embodiments, an alkyl group includes 1 or 2 carbon atoms. Branched chain alkyl groups include at least 3 carbon atoms and typically include 3 to 7, or in some embodiments, 3 to 6 carbon atoms. An alkyl group having 1 to 6 carbon atoms may be referred to as a (C₁-C₆)alkyl group or alternatively as a C₁-C₆ alkyl group having 1 to 4 carbon atoms may be referred to as a (C₁-C₄)alkyl or C₁-C₄ alkyl. This nomenclature may also be used for alkyl groups with differing numbers of carbon atoms. The term “alkyl may also be used when an alkyl group is a substituent that is further substituted in which case a bond between a second hydrogen atom and a C atom of the alkyl substituent is replaced with a bond to another atom such as, but not limited to, a halogen, or an O, N, or S atom. For example, a group —O—(C₁-C₆ alkyl)-OH will be recognized as a group where an —O atom is bonded to a C₁-C₆ alkyl group and one of the H atoms bonded to a C atom of the C₁-C₆ alkyl group is replaced with a bond to the O atom of an —OH group. As another example, a group —O—(C₁-C₆ alkyl)-O—(C₁-C₆ alkyl) will be recognized as a group where an —O atom is bonded to a first C₁-C₆ alkyl group and one of the H atoms bonded to a C atom of the first C₁-C₆ alkyl group is replaced with a bond to a second O atom that is bonded to a second C₁-C₆ alkyl group. Some alkyl groups may be referred to using names typically used with such groups. For example, a methyl group may be referred to as Me, and ethyl group may be referred to as Et, and a propyl group may be referred to as Pr.

“Alkenyl” refers to an unsaturated branched or straight-chain hydrocarbon group having at least one carbon-carbon double bond derived by the removal of one hydrogen atom from a single carbon atom of a parent alkene. The group may be in either the Z- or E-form (cis or trans) about the double bond(s). Typical alkenyl groups include, but are not limited to, ethenyl; propenyls such as prop-1-en-1-yl, prop-1-en-2-yl, prop-2-en-1-yl (allyl), and prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, and buta-1,3-dien-2-yl; and the like. In certain embodiments, an alkenyl group has 2 to 20 carbon atoms and in other embodiments, has 2 to 6 carbon atoms. An alkenyl group having 2 to 6 carbon atoms may be referred to as a (C₂-C₆) alkenyl group. The carbon atoms of an alkenyl group that are double bonded to one another are categorized as sp² hybridized carbon atoms.

“Alkoxy” refers to a group of formula —OR where R represents an alkyl group as defined herein. Representative examples include, but are not limited to, methoxy, ethoxy, propoxy, butoxy, and the like. Typical alkoxy groups include 1 to 10 carbon atoms, 1 to 6 carbon atoms or 1 to 4 carbon atoms in the R group. Alkoxy groups that include 1 to 6 carbon atoms may be designated as —O—(C₁-C₆) alkyl or as —O—(C₁-C₆ alkyl) groups. In some embodiments, an alkoxy group may include 1 to 4 carbon atoms and may be designated as —O—(C₁-C₄) alkyl or as —O—(C₁-C₄ alkyl) groups group. Alkoxy groups such as methoxy, ethoxy, and the like may be referred to respectively as OMe or OEt.

“Aryl” refers to a monovalent aromatic hydrocarbon group derived by the removal of one hydrogen atom from a single carbon atom of a parent aromatic ring system. Aryl encompasses monocyclic carbocyclic aromatic rings, for example, benzene. Aryl also encompasses bicyclic carbocyclic aromatic ring systems where each of the rings is aromatic, for example, naphthalene. Aryl groups may thus include fused ring systems where each ring is a carbocyclic aromatic ring. In certain embodiments, an aryl group includes 6 to 10 carbon atoms. Such groups may be referred to as C₆-C₁₀ aryl groups. Aryl, however, does not encompass or overlap in any way with heteroaryl as separately defined below. Hence, if one or more carbocyclic aromatic rings is fused with an aromatic ring that includes at least one heteroatom, the resulting ring system is a heteroaryl group, not an aryl group, as defined herein.

“Carbonyl” refers to a group of formula —C(O) which may also be referred to as —C(═O) group.

“Carboxy” refers to a group of formula —C(O)OH which may also be referred to as —C(═O)OH.

“Cyano” refers to a group of formula —CN.

“Cycloalkyl” refers to a saturated cyclic alkyl group derived by the removal of one hydrogen atom from a single carbon atom of a parent cycloalkane. Typical cycloalkyl groups include, but are not limited to, groups derived from cyclopropane, cyclobutane, cyclopentane, cyclohexane, cycloheptane, cyclooctane, and the like. Cycloalkyl groups may be described by the number of carbon atoms in the ring. For example, a cycloalkyl group having 3 to 8 ring members may be referred to as a (C₃-C₅)cycloalkyl, a cycloalkyl group having 3 to 7 ring members may be referred to as a (C₃-C₇)cycloalkyl and a cycloalkyl group having 4 to 7 ring members may be referred to as a (C₄-C₇)cycloalkyl. In certain embodiments, the cycloalkyl group can be a (C₃-C₁₀)cycloalkyl, a (C₃-C₅)cycloalkyl, a (C₃-C₇)cycloalkyl, a (C₃-C₆)cycloalkyl, or a (C₄-C₇)cycloalkyl group and these may be referred to as C₃-C₁₀ cycloalkyl, C₃-C₅ cycloalkyl, C₃-C₇ cycloalkyl, C₃-C₆ cycloalkyl, or C₄-C₇ cycloalkyl groups using alternative language. Cycloalkyl groups may be monocyclic or polycyclic. For the purposes of this application, the term “polycyclic” when used with respect to cycloalkyl will include bicyclic cycloalkyl groups such as, but not limited to, norbornane, bicyclo[1.1.1]pentane, and bicyclo[3.1.0]hexane, and cycloalkyl groups with more ring systems such as, but not limited to, cubane. The term “polycyclic” when used with respect to cycloalkyl will also include spirocyclic ring systems such as, but not limited to, spiro[2.2]pentane, spiro[2.3]hexane, spiro[3.3]heptane, and spiro[3.4]octane.

“Halo” or “halogen” refers to a fluoro, chloro, bromo, or iodo group.

“Haloalkyl” refers to an alkyl group in which at least one hydrogen is replaced with a halogen. Thus, the term “haloalkyl” includes monohaloalkyl (alkyl substituted with one halogen atom) and polyhaloalkyl (alkyl substituted with two or more halogen atoms). Representative “haloalkyl” groups include difluoromethyl, 2,2,2-trifluoroethyl, 2,2,2-trichloroethyl, and the like. The term “perhaloalkyl” means, unless otherwise stated, a haloalkyl group in which each of the hydrogen atoms is replaced with a halogen atom. For example, the term “perhaloalkyl”, includes, but is not limited to, trifluoromethyl, pentachloroethyl, 1,1,1-trifluoro-2-bromo-2-chloroethyl, and the like.

“Pharmaceutically acceptable” refers to generally recognized for use in animals, and more particularly in humans.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Stereoisomer” refers to an isomer that differs in the arrangement of the constituent atoms in space. Stereoisomers that are mirror images of each other and optically active are termed “enantiomers,” and stereoisomers that are not mirror images of one another and are optically active are termed “diastereomers.”

Provided herein are processes for synthesizing Mcl-1 inhibitors and intermediates useful in synthesizing Mcl-1 inhibitors. In particular, processes are set forth for synthesizing intermediates that may be used to prepare (1S,3′R,6′R,7′S,8′E,11′S,12′R)-6-chloro-7′-methoxy-11′,12′-dimethyl-3,4-dihydro-2H,15′H-spiro[naphthalene-1,22′[20]oxa[13]thia[1,14]diazatetracyclo[14.7.2.0^{3,6}0.0^{19,24}]pentacosa[8,16,18,24]tetraen]-15′-one 13′,13′-dioxide (compound A1), or a salt or solvate thereof, and for preparing (1S,3′R,6′R,7′R,8′E,11′S,12′R)-6-chloro-7′-methoxy-11′,12′-dinethyl-7′-((9aR)-octahydro-2H-pyrido[1,2-a]pyrazin-2-ylmethyl)-3,4-dihydro-2H,15′H-spiro[naphthalene-1,22′-[20]oxa[13]thia[1,14]diazatetracyclo[14.7.2.0^{3,6}0.0^{19,24}]pentacosa[8,16,18,24]-tetraen]-15′-one 13′,13′-dioxide (compound A2), or a salt or solvate thereof are provided. In some embodiments of the methods for synthesizing Compounds A1 and A2, the process provides a salt of the compound which may be a pharmaceutically acceptable salt. Compounds A1 and A2 are set forth below:

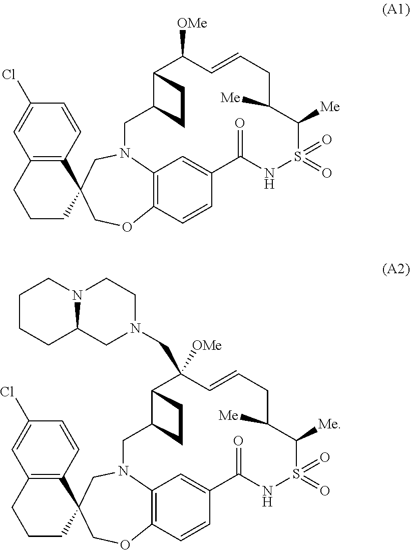

U.S. Pat. No. 9,562,061, which is incorporated herein by reference in its entirety, discloses compound A1, or a salt or solvate thereof, as an Mcl-1 inhibitor and provides a process for preparing it.

U.S. Pat. No. 10,300,075, which is incorporated herein by reference in its entirety, discloses compound A2, or a salt or solvate thereof, as an Mcl-1 inhibitor and provides a process for preparing it. The disclosure of compound A2 salts and solvates from U.S. Pat. No. 10,300,075 is incorporated by reference in its entirety.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### Embodiments

The embodiments listed below are presented in numbered form for convenience and in ease and clarity of reference in referring to multiple embodiments.

In embodiment 1, the invention provides a method of synthesizing a compound of Formula BI, the compound of Formula BI having the formula

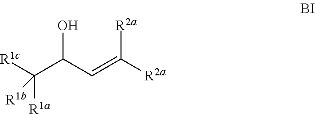

wherein the method comprises:
a) reacting a compound of Formula BII with an alkenyl boron compound and a catalyst in the presence of a base and optionally a solvent to form a product mixture comprising the compound of Formula BI, wherein the catalyst is prepared from a copper I salt or a copper II salt and a phosphine;
wherein the phosphine is at least two equivalents of a monophosphine or at least one equivalent of a diphosphine with respect to the copper I salt or is at least four equivalents of a monophosphine or at least two equivalents of a disphosphine with respect to the copper II salt;
and further wherein the sp² hybridized carbon atom of the alkenyl group that is not directly bonded to the boron atom of the alkenyl boron compound is bonded to 2 R^{2a} groups, wherein each R^{2a} is independently selected from —H, —C₁-C₆ alkyl, or a —C₆-C₁₀ aryl group, wherein the aryl group is unsubstituted or is substituted with 1 or 2 R^{3a} groups independently selected from —C₁-C₆ alkyl, —NO₂, halo, or —O—C₁-C₆; and further wherein if one of the R^{2a} groups is an aryl group, then the other R^{2a} group is not an aryl group;
wherein, BII has the formula

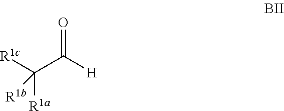

wherein
R^{1a}, R^{1b} and R^{1c} are independently selected from —H, or —C₁-C₆ alkyl, or OR^{1d} wherein R^{1d} is selected from —H, —C₁-C₆ alkyl, —Si(C₁-C₆ alkyl)₃, or C₁-C₆ alkyl-aryl, wherein the aryl in an R^{1d} group is a C₆-C₁₀ aromatic ring that is unsubstituted, or is substituted with 1, 2, or 3 substituents selected from —OH, —O—C₁-C₆ alkyl, —O—Si(C₁-C₆ alkyl)₃, halo, or C₁-C₆ haloalkyl;
or R^{1a} and R^{1b} may join to form a ring comprising 3, 4, 5, 6, 7, or 8 ring members comprising 0 or 1 oxygen atom, wherein the ring is unsubstituted or is substituted with 1 or 2 substituents selected from —OR¹ or —C₁-C₆—OR^{1e};
R^{1e} is selected from —H, —C₁-C₆ alkyl, —CH₂-aryl, —Si(C₁-C₆ alkyl)₃, tetrahydropyranyl, aryl, or —C═O—C₁-C₆ alkyl, wherein the aryl of an R^{1e} group is a C₆-C₁₀ aromatic ring that is unsubstituted or is substituted with 1, 2, or 3 substituents selected from —OR^{1f}, -halo, —C₁-C₆ alkyl, —C₁-C₆ haloalkyl, or —C═O—C₁-C₆ alkyl;
R^{1f} is selected from —H, —C₁-C₆ alkyl, —Si(C₁-C₆ alkyl)₃, tetrahydropyranyl, or —(C₁-C₆ alkyl)-aryl, wherein the aryl of an R^{1f} group is a C₆-C₁₀ aromatic ring that is unsubstituted, or is substituted with 1, 2, or 3 substituents selected from —OH, —O—C₁-C₆ alkyl, —O—Si(C₁-C₆ alkyl)₃, halo, or C₁-C₆ haloalkyl; and
b) reacting the product mixture with an oxidizing agent to oxidize the phosphine moieties in the phosphine to phosphine oxides to produce an oxidized phosphine.

In embodiment 2, the invention provides the method of embodiment 1, wherein the method further comprises: separating crystals of the oxidized phosphine from the reaction mixture.

In embodiment 3, the invention provides the method of embodiment 1 or embodiment 2 wherein the oxidizing agent is selected from H₂O₂, HOF, Ru(III)/O₂, or NaOCl.

In embodiment 4, the invention provides the method of embodiment 3, wherein the oxidizing agent is an aqueous solution of H₂O₂.

In embodiment 5, the invention provides the method of embodiment 2, wherein the method further comprises: reacting the separated oxidized phosphine oxide with a reducing agent to provide the phosphine.

In embodiment 6, the invention provides the method of embodiment 5, wherein the reducing agent is selected from HSiCl₃, HSiCl₃:N(C₁-C₆ alkyl)₃, Si₂Cl₆, PhSiH₃, Ph₂SiH₂, Me₃SiH, Et₃SiH, PhMe₂SiH, Ph₃SiH, (Me₃Si)₃Si—H, PhCH₂SiH₃, naphthylsilane, bis(naphthyl)silane, bis(4-methylphenyl)silane, bis(fluorenyl)silane, HSi(OEt)₃, HSi(OEt)₃ with Ti(C₁-C₆ alkoxide)₄, 1,3-diphenyldisiloxane, hexamethyldisilane, TfOSi(H)(CH₃)₂, (CH₃)₂Si(H)—O—Si(CH₃)₂(H) with Cu(OTf)₂, tetramethyldisiloxane, polymethylhydrosiloxane, dialkylphosphite with I₂ and P(OPh)₃, AlH₃ with diisobutylaluminum hydride, or borane reducing agents, wherein Tf is triflate.

In embodiment 7, the invention provides the method of embodiment 6, wherein the reducing agent is HSiCl₃.

In embodiment 8, the invention provides the method of any one of embodiments 1-7, wherein R^{1a} and R^{1b} join to form a substituted or unsubstituted ring with 3, 4, 5, or 6 ring members each of which is a carbon atom.

In embodiment 9, the invention provides the method of embodiment 8, wherein R^{1a} and R^{1b} join to form a substituted or unsubstituted ring with 4 ring members each of which is a carbon atom.

In embodiment 10, the invention provides the method of embodiment 8 or embodiment 9, wherein R^{1e} is —H.

In embodiment 11, the invention provides the method of any one of embodiments 8-10, wherein R^{1a} and R^{1b} join to form a 4 membered ring that is substituted with 1 —C₁-C₆—OR^{1e} substituent.

In embodiment 12, the invention provides the method of embodiment 1, wherein R^{1a} and R^{1b} join to form a 4 membered ring that is substituted with 1 —CH₂—OR^{1e} substituent.

In embodiment 13, the invention provides the method of embodiment 12, wherein R^{1a} and R^{1b} join to form a 4 membered ring that is substituted with 1 —CH₂—O—C═O—C₁-C₆ alkyl substituent.

In embodiment 14, the invention provides the method of embodiment 13, wherein R^{1a} and R^{1b} join to form a 4 membered ring that is substituted with 1 —CH₂—O—C═O—CH₃ substituent.

In embodiment 15, the invention provides the method of any one of embodiments 1-8, wherein the compound of formula BI, has the formula IA

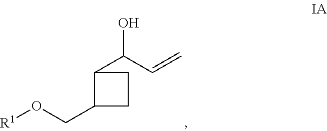

wherein R¹ is a —C═O—C₁-C₆ alkyl group.

In embodiment 16, the invention provides the method of embodiment 15, wherein the compound of Formula BI has the Formula IB

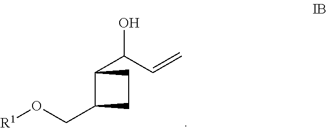

In embodiment 17, the invention provides the method of embodiment 15, wherein the compound of Formula IA has the Formula IC

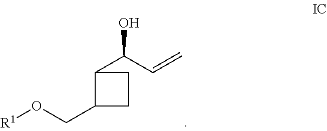

In embodiment 18, the invention provides the method of embodiment 15, wherein the compound of Formula BI has the Formula ID

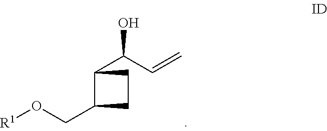

In embodiment 19, the invention provides the method of embodiment 15, wherein the compound of Formula BI has the Formula IE

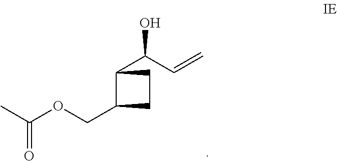

In embodiment 20, the invention provides the method of embodiment 15, wherein the compound of Formula BI is formed as a mixture of the compounds of Formula ID and ID′, wherein the compounds of formula ID and ID′ have the structures:

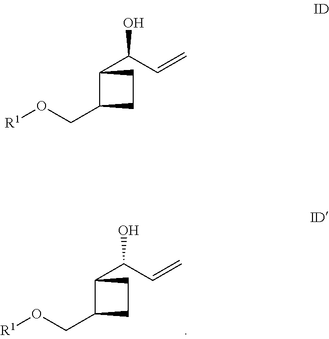

In embodiment 21, the invention provides the method of embodiment 20, wherein the amount of ID to ID′ or the amount of ID′ to ID ranges from 60:40 to 100:0.

In embodiment 22, the invention provides the method of embodiment 20, wherein the amount of ID to ID′ or the amount of ID′ to ID ranges from 65:35 to 99.9:0.1.

In embodiment 23, the invention provides the method of embodiment 20, wherein the amount of ID to ID′ or the amount of ID′ to ID ranges from 70:30 to 99.1:0.1.

In embodiment 24, the invention provides the method of embodiment 20, wherein the amount of ID to ID′ or the amount of ID′ to ID ranges from 75:25 to 99.9:0.1.

In embodiment 25, the invention provides the method of embodiment 20, wherein the percent of ID present in the mixture based on the total of the amount of ID and ID′ is 60% or greater.

In embodiment 26, the invention provides the method of embodiment 20, wherein the percent of ID present in the mixture based on the total of the amount of ID and ID′ is 70% or greater.

In embodiment 27, the invention provides the method of embodiment 20, wherein the percent of ID present in the mixture based on the total of the amount of ID and ID′ is 80% or greater.

In embodiment 28, the invention provides the method of embodiment 20, wherein the percent of ID present in the mixture based on the total of the amount of ID and ID′ is 85% or greater.

In embodiment 29, the invention provides the method of embodiment 20, wherein the percent of ID present in the mixture based on the total of the amount of ID and ID′ is 90% or greater.

In embodiment 30, the invention provides the method of embodiment 20, wherein the percent of ID present in the mixture based on the total of the amount of ID and ID′ is 95% or greater.

In embodiment 31, the invention provides the method of embodiment 20, wherein the compound of Formula ID has the structure IE and the compound of ID′ has the structure IE′,

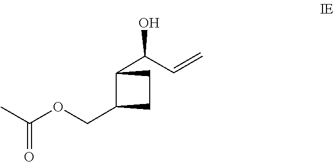

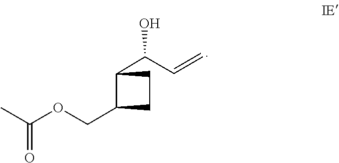

In embodiment 32, the invention provides the method of any one of embodiments 1-19, wherein the phosphine has at least one chiral center.

In embodiment 33, the invention provides the method of any one of embodiments 1-32, wherein the phosphine is a monophosphine.

In embodiment 34, the invention provides the method of any one of embodiments 1-32, wherein the phosphine is a diphosphine.

In embodiment 35, the invention provides the method of any one of embodiments 1-34, wherein the phosphine is selected from (R)-(+)-2,2′-bis(diphenylphosphino)-1,1′-binaphthyl ((R)-BINAP), 4(R)-(4,4′-bi-1,3-benzodioxole)-5,5′-diyl]bis[diphenylphosphine] ((R)-SEGPHOS), 1,1′-ferrocenediyl-bis(diphenylphosphine) (dppf), 1,3-bis(diphenylphosphino)propane (dppp), 1,2-bis(diphenylphosphino)ethane (dppe), PPh₃, 2,2′-bis(di-p-tolylphosphino)-1,1′-binaphthyl (TolBINAP), 2,2-bis[di(3,5-xylyl)phosphino]-1,1′-binaphthyl (XylBINAP), 5,5′-bis[di(3,5-xylyl)phosphino]-4,4′-bi-1,3-benzodioxole (DM-SEGPHOS), or (R)-1,13-bis(diphenylphosphino)-7,8-dihydro-6H-dibenzo[f,h][1,5]dioxonin ((R)-C3-TunePhos). In other embodiments, the phosphine is selected from 2,2′-bis(diphenylphosphino)-1,1′-binaphthyl (BINAP), 4,4′-bi-1,3-benzodioxole-5,5′-diylbis(diphenylphosphane) (SEGPHOS), 1,1′-ferrocenediyl-bis(diphenylphosphine) (dppf), 1,3-bis(diphenylphosphino)propane (dppp), 1,2-bis(diphenylphosphino)ethane (dppe), PPh₃, 2,2′-bis(di-p-tolylphosphino)-1,1′-binaphthyl (TolBINAP), 2-2′-bis[di3,5-xylyl)phosphino]-1,1′-binaphthyl (XylBINAP), 5,5′-bis[di(3,5-xylyl)phosphino]-4,4′-bi-1,3-benzodioxole (DM-SEGPHOS), or 1,13-bis(diphenylphosphino)-7,8-dihydro-6H-dibenzo[f,h][1,5]dioxonin (C3-TunePhos). In some embodiments, the phosphine is selected from (S)-(−)-2,2′-bis(diphenylphosphino)-1,1′-binaphthyl ((S)-BINAP), 4(S)-(4,4′-bi-1,3-benzodioxole)-5,5′-diyl]bis[diphenylphosphine] ((S)-SEGPHOS), 1,1′-ferrocenediyl-bis(diphenylphosphine) (dppf), 1,3-bis(diphenylphosphino)propane (dppp), 1,2-bis(diphenylphosphino)ethane (dppe), PPh₃, 2,2′-bis(di-p-tolylphosphino)-1,1′-binaphthyl (TolBINAP), 2,2-bis[di(3,5-xylyl)phosphino]-1,1′-binaphthyl (XylBINAP), 5,5′-bis[di(3,5-xylyl)phosphino]-4,4′-bi-1,3-benzodioxole (DM-SEGPHOS), or (S)-1,13-bis(diphenylphosphino)-7,8-dihydro-6H-dibenzo[f,h][1,5]dioxonin ((S)-C3-TunePhos).

In embodiment 36, the invention provides the method of any one of embodiments 1-32, wherein the phosphine is (R)-DTBM-SEGPHOS having the structure

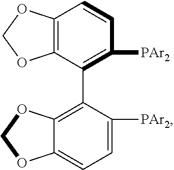

wherein Ar has the structure

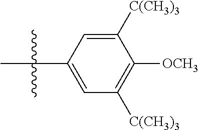 [SMILES: *c1cc(C(C)(C)C)c(OC)c(C(C)(C)C)c1]

wherein the [symbol] indicates the point of attachment to the rest of the molecule.

In some embodiments, the catalyst is prepared from a copper I salt whereas in other embodiments, the catalyst is prepared from a copper II salt. In embodiment 37, the invention provides the method of any one of embodiments 1-36, wherein the copper I salt or copper II salt is selected from copper(I) hexafluorophosphate, copper(I) tetrafluoroborate, CuF(PPh₃)₃, CuF₂, CuF, CuI, Cu(OTf)₂, or Cu(OTf), wherein Tf is triflate.

In embodiment 38, the invention provides the method of any one of embodiments 1-36, wherein the copper I salt is used to prepare the catalyst and the copper I salt is copper(I) hexafluorophosphate or copper(I) tetrafluoroborate.

In embodiment 39, the invention provides the method of any one of embodiments 1-38, wherein the alkenyl boron compound is selected from 4,4,5,5-tetramethyl-2-vinyl-1,3,2-dioxaborolane. vinylBF₃K, 4,4,6-trimethyl-2-vinyl-1,3,2-dioxaborinane, vinylB(OH)₂, vinylboronic anhydride, vinylboronic acid MIDA ester, (E)-4,4,5,5-tetramethyl-2-styryl-1,3,2-dioxaborolane, (E)-4,4,5,5-tetramethyl-2-(prop-1-en-1-yl)-1,3,2-dioxaborolane, (E)-2-(3,3-dimethylbut-1-en-1-yl)-4,4,5,5-tetramethyl-1,3,2-dioxaborolane, or (E)-4,4,5,5-tetramethyl-2-(oct-1-en-1-yl)-1,3,2-dioxaborolane.

In embodiment 40, the invention provides the method of any one of embodiments 1-38, wherein the alkenyl boron compound is

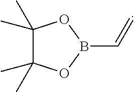

The reaction may be run without a solvent. However, the reaction gives improved yields when a solvent is used. Thus, In embodiment 41, the invention provides the method of any one of embodiments 1-40, wherein the reaction is conducted in the presence of an organic solvent.

In embodiment 42, the invention provides the method of embodiment 41, wherein the solvent is selected from isopropyl acetate, toluene, ethyl acetate, xylene, 2-methyltetrahydrofuran, tetrahydrofuran, cyclopentyl methyl ether, or t-butyl methyl ether. Various other solvents may be employed in accordance with the invention.

In embodiment 43, the invention provides the method of any one of embodiments 1-40, wherein the reaction is conducted in a solvent and the solvent is isopropyl acetate.

In embodiment 44, the invention provides the method of any one of embodiments 1-43, wherein the compound of Formula BII is reacted with the alkenyl boron compound and the catalyst in the presence of the base and the base is selected from K₃PO₄. CsF, Cs₂CO₃, Na₂CO₃, K₂CO₃, NaF, KF, Na₃PO₄, or Cs₃PO₄.

In embodiment 45, the invention provides the method of any one of embodiments 1-43, wherein the compound of Formula BII is reacted with the alkenyl boron compound and the catalyst in the presence of the base and the base is K₃PO₄.

In embodiment 46, the invention provides the method of any one of embodiments 1-45, wherein the compound of Formula BII is reacted with the alkenyl boron compound and the catalyst at a temperature ranging from 15° C. to 50° C.

In embodiment 47, the invention provides the method of embodiment 46, wherein the compound of Formula BII is reacted with the alkenyl boron compound and the catalyst at a temperature ranging from 20° C. to 40° C.

In embodiment 48, the invention provides a method of synthesizing a compound of Formula IA′, the compound of Formula IA′ having the formula:

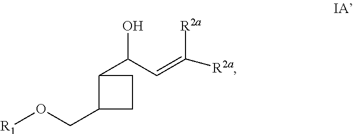

wherein the method comprises:
reacting a compound of Formula IIA with a alkenyl boron compound and a catalyst in the presence of a base and an optional solvent to form a product mixture comprising the compound of Formula IA′, wherein the catalyst is prepared from a copper I salt or a copper II salt and a phosphine, wherein the phosphine is at least two equivalents of a monophosphine or at least one equivalent of a diphosphine with respect to the copper I salt, or is at least four equivalents of a monophosphine or at least two equivalents of a disphosphine with respect to the copper II salt,
and further wherein the sp² hybridized carbon atom of the alkenyl group that is not directly bonded to the boron atom of the alkenyl boron compound is bonded to 2 R^{2a} groups, wherein each R^{2a} is independently selected from —H, —C₁-C₆ alkyl, or a —C₆-C₁₀ aryl group, wherein the aryl group is unsubstituted or is substituted with 1 or 2 R^{3a} groups independently selected from —C₁-C₆ alkyl, —NO₂, halo, or —O—C₁-C₆ alkyl; and further wherein if one of the R^{2a} groups is an aryl group, then the other R^{2a} group is not an aryl group;
wherein the compound of Formula IIA has the structure

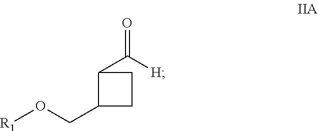

wherein R¹ is selected from —H, —C₁-C₆ alkyl, —C═O—C₁-C₆ alkyl, —C═O-aryl, —Si(C₁-C₆ alkyl)₃, tetrahydropyranyl, or —C₁-C₆ alkyl-aryl, wherein the aryl group in an R¹ group is a C₆-C₁₀ aromatic ring that is unsubstituted, or is substituted with 1, 2, or 3 substituents selected from —OH, NO₂, —O—C₁-C₆ alkyl, halo, or C₁-C₆ haloalkyl.

In embodiment 49, the invention provides the method of embodiment 48, wherein the compound of Formula IA′ has the Formula IA

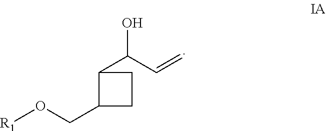

In embodiment 50, the invention provides the method of embodiment 49, wherein the compound of Formula IA′ has the Formula IB

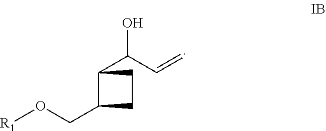

In embodiment 51, the invention provides the method of embodiment 49, wherein the compound of Formula IA′ has the Formula IC

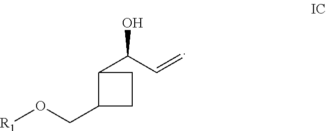

In embodiment 52, the invention provides the method of embodiment 49, wherein the compound of Formula IA′ has the Formula ID

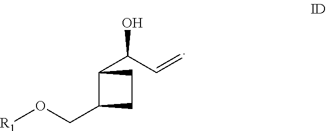

In embodiment 53, the invention provides the method of embodiment 49, wherein the compound of Formula IA′ has the Formula IE

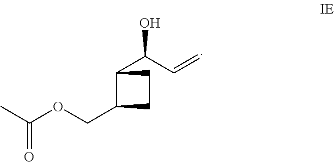

In embodiment 54, the invention provides the method of embodiment 49, wherein the compound of Formula IA′ is formed as a mixture of the compounds of Formula ID and ID′, wherein the compounds of Formula ID and Formula ID′ have the structures:

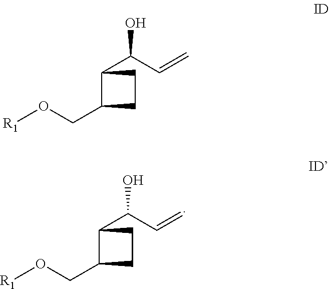

In embodiment 55, the invention provides the method of embodiment 54, wherein the amount of ID to ID′ or the amount of ID′ to ID ranges from 60:40 to 100:0.

In embodiment 56, the invention provides the method of embodiment 54, wherein the amount of ID to ID′ or the amount of ID′ to ID ranges from 65:35 to 99.9:0.1.

In embodiment 57, the invention provides the method of embodiment 54, wherein the amount of ID to ID′ or the amount of ID′ to ID ranges from 70:30 to 99.1:0.1.

In embodiment 58, the invention provides the method of embodiment 54, wherein the amount of ID to ID′ or the amount of ID′ to ID ranges from 75:25 to 99.9:0.1.

In embodiment 59, the invention provides the method of embodiment 54, wherein the percent of ID present in the mixture based on the total of the amount of ID and ID′ is 60% or greater.

In embodiment 60, the invention provides the method of embodiment 54, wherein the percent of ID present in the mixture based on the total of the amount of ID and ID′ is 70% or greater.

In embodiment 61, the invention provides the method of embodiment 54, wherein the percent of ID present in the mixture based on the total of the amount of ID and ID′ is 80% or greater.

In embodiment 62, the invention provides the method of embodiment 54, wherein the percent of ID present in the mixture based on the total of the amount of ID and ID′ is 85% or greater.

In embodiment 63, the invention provides the method of embodiment 54, wherein the percent of ID present in the mixture based on the total of the amount of ID and ID′ is 90% or greater.

In embodiment 64, the invention provides the method of embodiment 54, wherein the percent of ID present in the mixture based on the total of the amount of ID and ID′ is 95% or greater.

In embodiment 65, the invention provides the method of embodiment 54, wherein the compound of Formula ID has the structure IE and the compound of ID′ has the structure IE′,

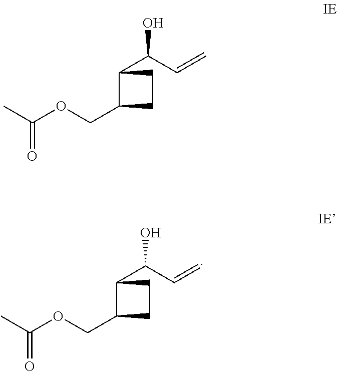

In embodiment 66, the invention provides the method of any one of embodiments 48-65, wherein the phosphine has at least one chiral center.

In embodiment 67, the invention provides the method of any one of embodiments 48-65, wherein the phosphine is a monophosphine.

In embodiment 68, the invention provides the method of any one of embodiments 48-65, wherein the phosphine is a diphosphine.

In embodiment 69, the invention provides the method of any one of embodiments 48-65, wherein the phosphine is selected from (R)-(+)-2,2′-bis(diphenylphosphino)-1,1′-binaphthyl ((R)-BINAP), 4(R)-(4,4′-bi-1,3-benzodioxole)-5,5′-diyl]bis[diphenylphosphine] ((R)-SEGPHOS), 1,1′-ferrocenediyl-bis(diphenylphosphine) (dppf), 1,3-bis(diphenylphosphino)propane (dppp), 1,2-bis(diphenylphosphino)ethane (dppe), PPh₃, 2,2′-bis(di-p-tolylphosphino)-1,1′-binaphthyl (TolBINAP), 2,2-bis[di(3,5-xlyl)phosphino]-1,1′-binaphthyl (XylBINAP), 5,5′-bis[di(3,5-xylyl)phosphino]-4,4′-bi-1,3-benzodioxole (DM-SEGPHOS), or (R)-1,13-bis(diphenylphosphino)-7,8-dihydro-6H-dibenzo[f,h][1,5]dioxonin ((R)-C3-TunePhos).

In embodiment 70, the invention provides the method of any one of embodiments 48-65, wherein the phosphine is (R)-DTBM-SEGPHOS having the structure

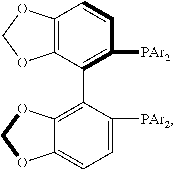

wherein Ar has the structure

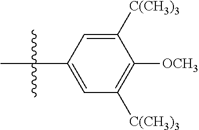 [SMILES: *c1cc(C(C)(C)C)c(OC)c(C(C)(C)C)c1]

wherein the [symbol] indicates the point of attachment to the rest of the molecule.

In some embodiments, the catalyst is prepared from a copper I salt whereas in other embodiments, the catalyst is prepared from a copper II salt. In embodiment 71, the invention provides the method of any one of embodiments 48-70, wherein the copper I salt or copper II salt is selected from copper(I) hexafluorophosphate, copper(I) tetrafluoroborate, CuF(PPh₃)₃, CuF₂, CuF, CuI, Cu(OTf)₂, or Cu(OTf), wherein Tf is triflate.

In embodiment 72, the invention provides the method of any one of embodiments 48-70, wherein catalyst is prepared from a copper I salt and the copper I salt is copper(I) hexafluorophosphate or copper(I) tetrafluoroborate.

In embodiment 73, the invention provides the method of any one of embodiments 48-72, wherein the alkenyl boron compound is selected from 4,4,5,5-tetramethyl-2-vinyl-1,3,2-dioxaborolane. vinylBF₃K, 4,4,6-trimethyl-2-vinyl-1,3,2-dioxaborinane, vinylB(OH)₂, vinylboronic anhydride, vinylboronic acid MIDA ester, (E)-4,4,5,5-tetramethyl-2-styryl-1,3,2-dioxaborolane, (E)-4,4,5,5-tetramethyl-2-(prop-1-en-1-yl)-1,3,2-dioxaborolane, (E)-2-(3,3-dimethylbut-1-en-1-yl)-4,4,5,5-tetramethyl-1,3,2-dioxaborolane, or (E)-4,4,5,5-tetramethyl-2-(oct-1-en-1-yl)-1,3,2-dioxaborolane.

In embodiment 74, the invention provides the method of any one of embodiments 48-72, wherein the alkenyl boron compound is

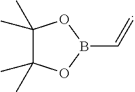

In embodiment 75, the invention provides the method of any one of embodiments 48-74, wherein the compound of Formula IIA is reacted with the alkenyl boron compound and the catalyst in the presence of the base and the solvent, wherein the solvent is selected from isopropyl acetate, toluene, ethyl acetate, xylene, 2-methyltetrahydrofuran, tetrahydrofuran, cyclopentyl methyl ether, or t-butyl methyl ether.

In embodiment 76, the invention provides the method of any one of embodiments 48-74, wherein the compound of Formula IIA is reacted with the alkenyl boron compound and the catalyst in the presence of the base and the solvent, wherein the solvent is isopropyl acetate.

In embodiment 77, the invention provides the method of any one of embodiments 48-76, wherein the base is selected from K₃PO₄, CsF, Cs₂CO₃, Na₂CO₃, K₂CO₃, NaF, KF, Na₃PO₄, or Cs₃PO₄.

In embodiment 78, the invention provides the method of any one of embodiments 48-76, wherein the base is K₃PO₄.

In embodiment 79, the invention provides the method of any one of embodiments 48-78, wherein the compound of Formula BII is reacted with the alkenyl boron compound and the catalyst at a temperature ranging from 15° C. to 50° C.

In embodiment 80, the invention provides the method of embodiment 79, wherein the compound of Formula BII is reacted with the alkenyl boron compound and the catalyst at a temperature ranging from 20° C. to 40° C.

In embodiment 81, the invention provides the method of any one of embodiments 48-80, wherein the method further comprises: reacting the product mixture with an oxidizing agent to oxidize the phosphine moieties in the phosphine to phosphine oxides to produce an oxidized phosphine.

In embodiment 82, the invention provides the method of embodiment 81, wherein the method further comprises separating crystals of the oxidized phosphine from the reaction mixture.

In embodiment 83, the invention provides the method of embodiment 81 or embodiment 82 wherein the oxidizing agent is selected from H₂O₂, HOF, Ru(III)/O₂, or NaOCl.

In embodiment 84, the invention provides the method of embodiment 81 or embodiment 82, wherein the oxidizing agent is an aqueous solution of H₂02.

In embodiment 85, the invention provides the method of any one of embodiments 82-84, wherein the method further comprises: reacting the separated oxidized phosphine with a reducing agent to provide the phosphine.

In embodiment 86, the invention provides the method of embodiment 85, wherein the reducing agent is selected from selected from HSiCl₃, HSiCl₃:N(C₁-C₆ alkyl)₃, Si₂Cl₆, PhSiH₃, Ph₂SiH₂, PhCH₂SiH₃, Me₃SiH, Et₃SiH, PhMe₂SiH, Ph₃SiH, (Me₃Si)₃Si—H, naphthylsilane, bis(naphthyl)silane, bis(4-methylphenyl)silane, bis(fluorenyl)silane, HSi(OEt)₃, HSi(OEt)₃ with Ti(C₁-C₆ alkoxide)₄, 1,3-diphenyldisiloxane, hexamethyldisilane, TfOSi(H)(CH₃)₂, (CH₃)₂Si(H)—O—Si(CH₃)₂(H) with Cu(OTf)₂, tetramethyldisiloxane, polymethylhydrosiloxane, dialkylphosphite with I₂ and P(OPh)₃, AlH₃ with diisobutylaluminum hydride, or borane reducing agents, wherein Tf is triflate.

In embodiment 87, the invention provides the method of embodiment 86, wherein the reducing agent is HSiCl₃.

In embodiment 88, the invention provides a compound of Formula IA having the formula

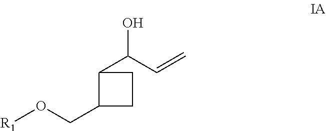

wherein R¹ is selected from —H, —C₁-C₆ alkyl, C═O—C₁-C₆ alkyl, —Si(C₁-C₆ alkyl)₃, tetrahydropyranyl, —C₁-C₆ alkyl-aryl, wherein the aryl in an R¹ group is a C₆-C₁₀ aromatic ring that is unsubstituted, or is substituted with 1, 2, or 3 substituents selected from —OH, —O—C₁-C₆ alkyl, halo, or C₁-C₆ haloalkyl.

In embodiment 89, the invention provides the compound of embodiment 88, wherein the compound of Formula IA has the Formula IB

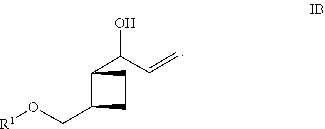

In embodiment 90, the invention provides the compound of embodiment 88, wherein the compound of Formula IA has the Formula IC

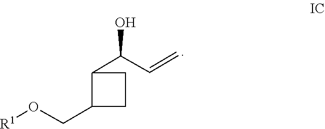

In embodiment 91, the invention provides the compound of embodiment 88, wherein the compound of Formula IA has the Formula ID

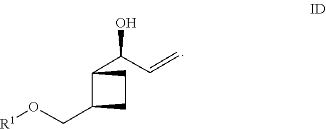

In embodiment 92, the invention provides the compound of embodiment 88, wherein the compound of Formula IA has the Formula IE

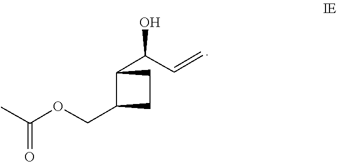

In embodiment 93, the invention provides a method for synthesizing compound A3 using compound IA according to any one of embodiments 15-47 or 48-87, wherein the compound A3 has the following structure:

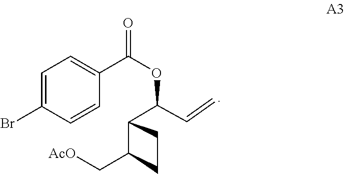 [SMILES: C=C[C@H](OC(=O)c1ccc(Br)cc1)[C@@H]1CC[C@H]1COC(C)=O]

In embodiment 94, the invention provides a method for synthesizing compound A1 using compound IA according to any one of embodiments 15-47 or 48-87, wherein the compound A1 has the following structure:

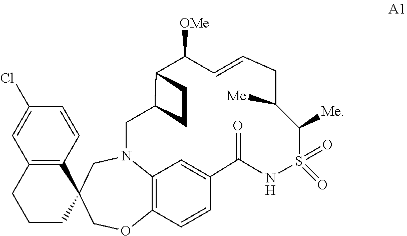

In embodiment 95, the invention provides a method for synthesizing compound A2 using compound IA according to any one of embodiments 15-47 or 48-87, wherein the compound A2 has the following structure:

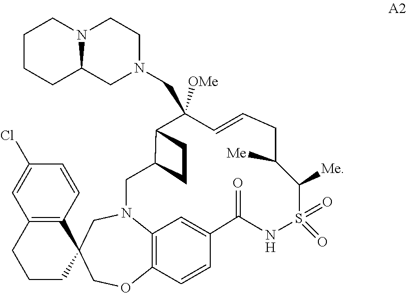

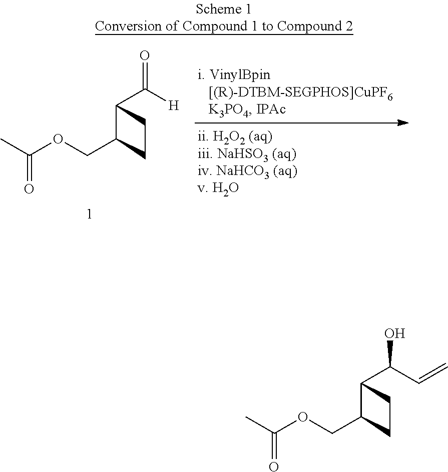

As shown in Scheme 1, reaction of an aldehyde such as Compound 1 with an alkenyl boron compound such as a vinyl boronate ester such 4,4,5,5-tetramethyl-2-vinyl-1,3,2-dioxaborolane (VinylBpin) with a catalyst comprising a phosphine ligand and a copper (I) salt leads to formation of a vinyl alcohol such as Compound 2 as the major product. Notably, the stereochemistry may be reversed at the carbon bearing the hydroxyl group in compound 2 using the enantiomer of (R)-DTBM-SEGPHOS or another chiral phosphine ligand as shown in Scheme 2. Similarly, a phosphine ligand without any chiral centers may be used to generate alcohol compounds if no specific stereochemistry at the carbon bearing the hydroxyl group is desired. It should be noted that choice of an appropriate optically active ligand may be used to control the stereochemistry of the major product with respect to the stereochemistry of the carbon bearing the hydroxyl group as shown in Schem 2.

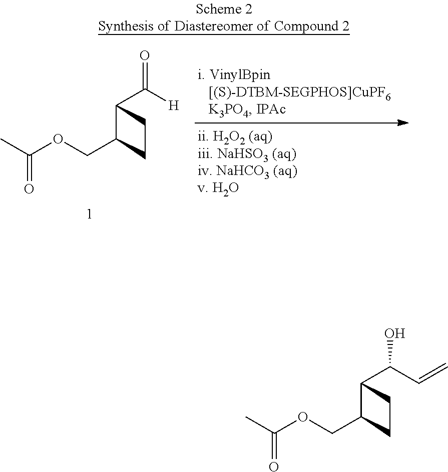

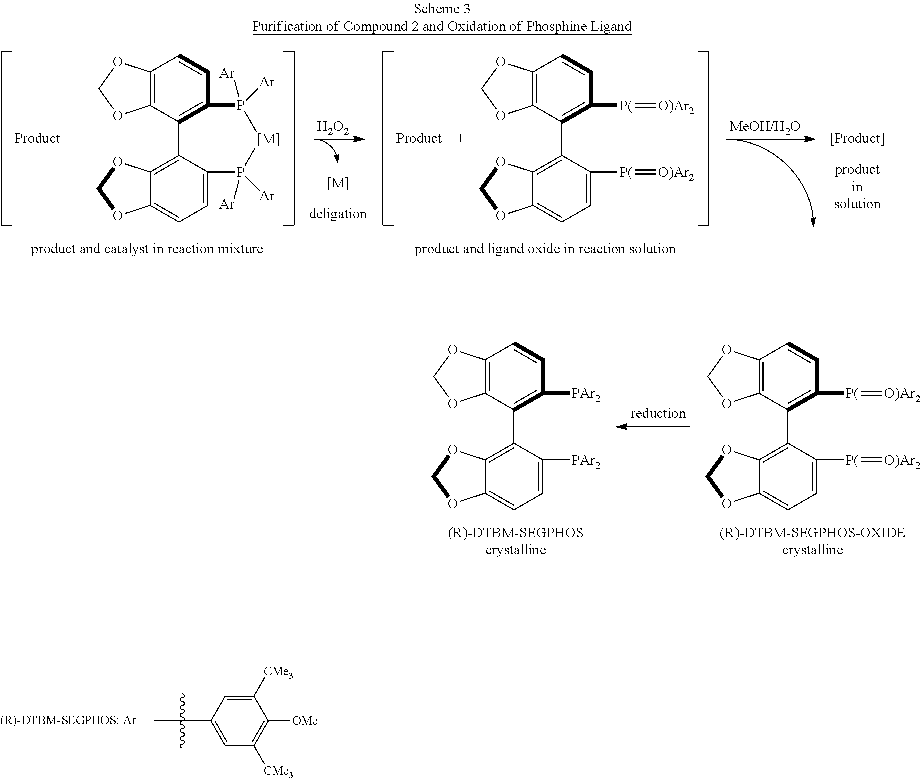

In practice, purification of the reaction product is simplified through adjusting the oxidation state of the catalyst/ligand through the approach outlined herein As shown in Scheme 3, a mixture of the reaction product and the catalyst is treated with an oxidizing agent such as hydrogen peroxide. The oxidation converts the phosphine to the crystalline phosphine oxide which can then be separated from the liquid alcohol reaction product by crystallization and filtration. The isolated phosphine oxide can then be reduced with reducing agents such as HSiCl₃ to provide the phosphine ligand. Because ligands such as (R)-DTBM-SEGPHOS are quite expensive, this methodology allows for purification of the alcohol reaction product and for regeneration of the phosphine ligand.

The processes for synthesizing compound 2 can be used to synthesize compounds A1 and A2 as shown in Scheme 4, Scheme 5, Scheme 6, and Scheme 7 below. As shown in Scheme 4, compound 2 may be used to synthesize compound 7 and salts and solvates thereof and compound 7 may be used to synthesize compound 10 as shown in Scheme 5. Compound 10 may then be used to synthesize compound A1 and salts and solvates thereof and compound A2 and salts and solvates thereof as shown in Scheme 6 and Scheme 7.

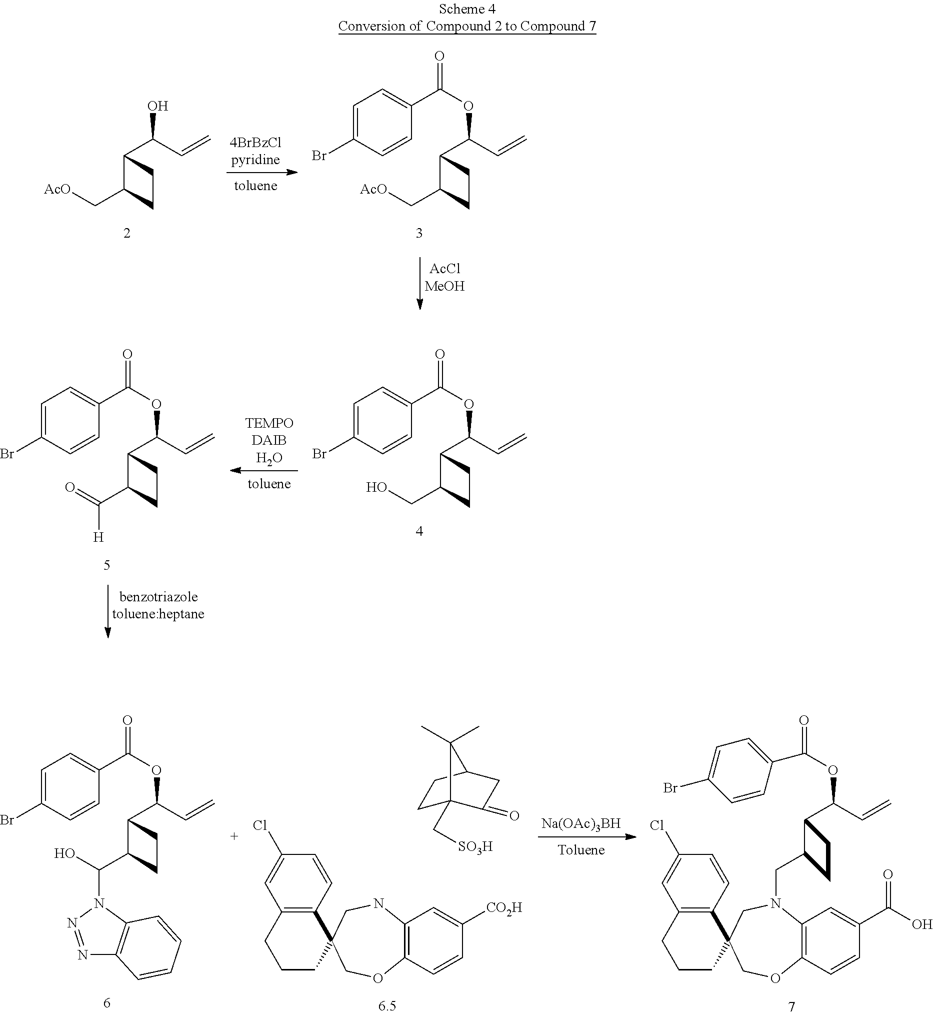

As shown in Scheme 4 and set forth in the Examples, compound 2 may be used to synthesize compound 7 and salts and solvates thereof. As described herein, compound 2 can be used to prepare compound 3 by reaction with 4-bromobenzoyl chloride. Removal of the acetate protecting group from compound 3 provides compound 4 which may be oxidized to provide compound 5. Reaction of 5 with benzotriazole provides compound 6. Compound 6.5 may be prepared using the procedures set forth in U.S. Pat. No. 9,562,061. Compound 6 and compound 6.5 can be reacted to form compound 7.

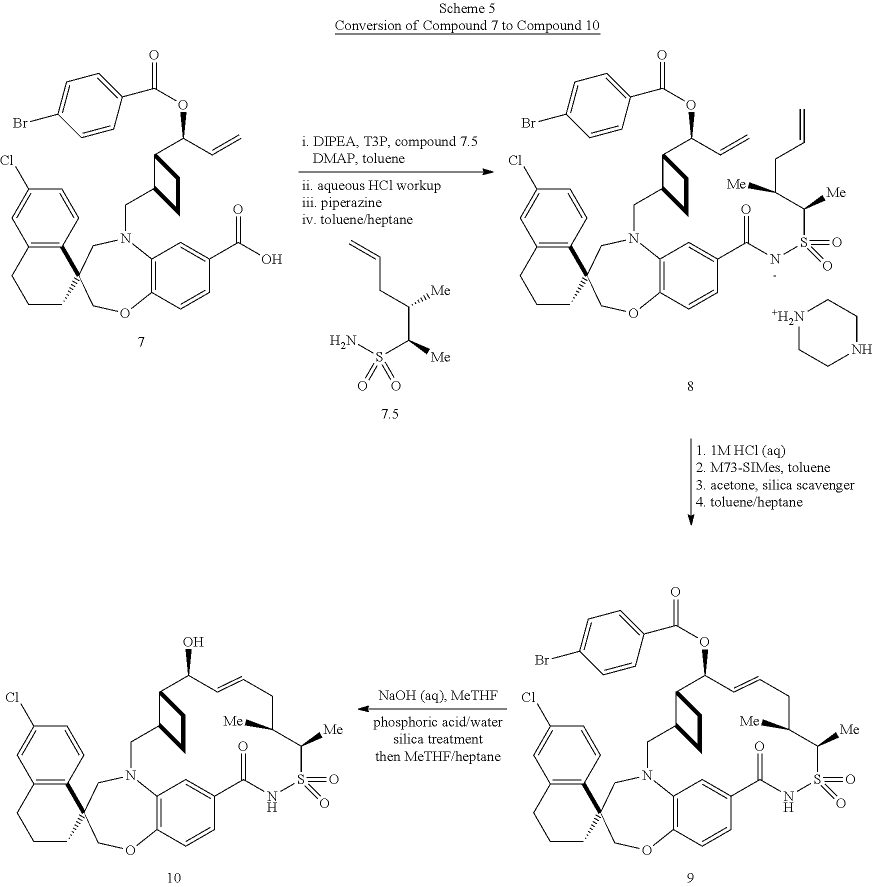

As shown in Scheme 5, compound 7 or salts or solvates thereof may be used to synthesize compound 10 and used to prepare compound A1 and salts and solvates thereof and compound A2 and salts and solvates thereof. The synthesis of sulfonamide 7.5 is disclosed in U.S. Pat. No. 9,562,061. As described herein, reaction of compound 7 with sulfonamide 7.5 can be used to prepare compound 8 which may then be cyclized to form compound 9. Removal of the protecting group from compound 9 provides hydroxy compound 10 which can then be converted to compound A1 and salts and solvates thereof and compound A2 and salts and solvates thereof as shown in Scheme 6 and Scheme 7.

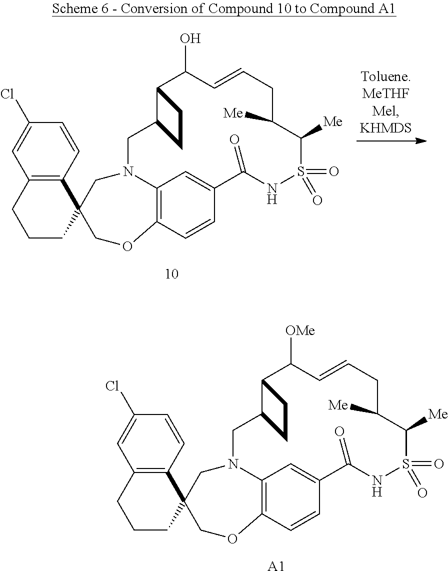

As shown in Scheme 5 and described in U.S. Pat. No. 9,562,061, compound 10 may be generated from compound 2 and so both compounds may be used to synthesize compound A1 and salts and solvates thereof. For example, as shown in Scheme 6, compound 10 may be methylated to provide compound A1 as described in U.S. Pat. No. 9,562,061 and set forth in Example 11.

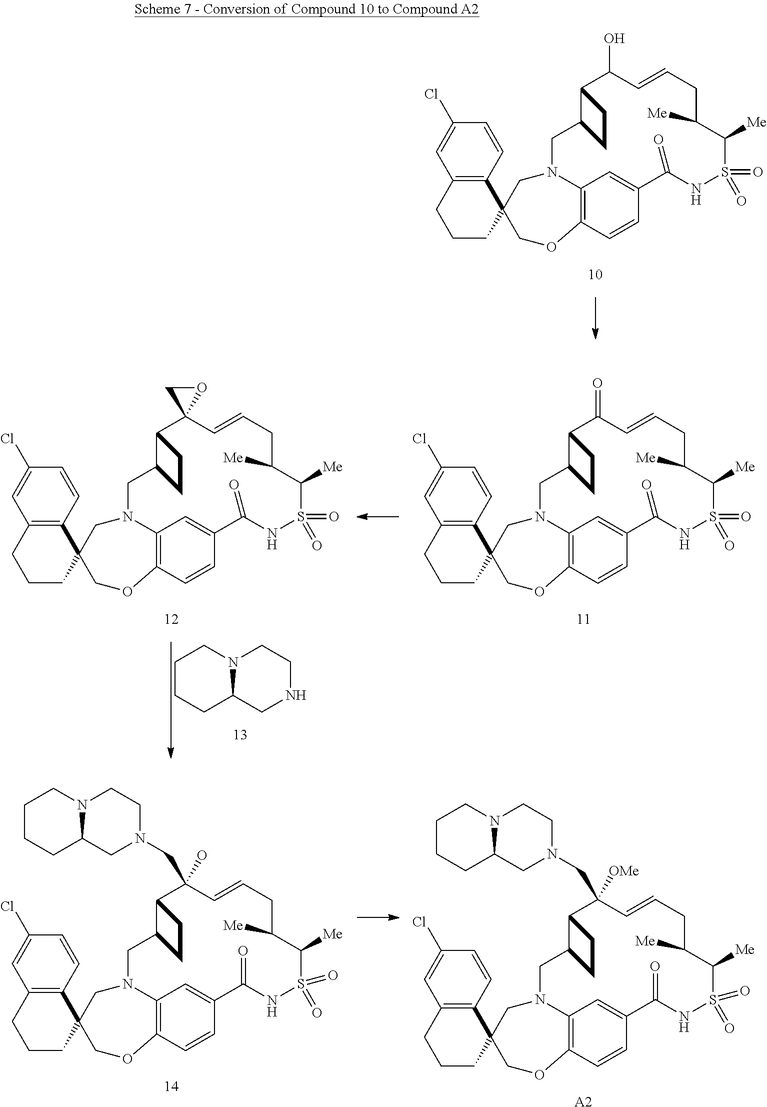

As shown in Scheme 7 and described in U.S. Pat. No. 10,300,075, compound 10, generated from compound 2, can also be used to synthesize compound A2 and salts and solvates thereof. As shown above, compound 10 can be oxidized to provide cyclic enone 11 using the methodology disclosed in U.S. Pat. No. 10,300,075. Enone 11 can then be converted to epoxide 12 using the procedures disclosed in U.S. Pat. No. 10,300,075. Epoxide 12 can then be reacted with bicyclic compound 13 to provide hydroxy compound 14. Finally, methylation of compound 14 can provide compound A2 as disclosed in U.S. Pat. No. 10,300,075.

In some embodiments, the processes further include synthesizing compound A1 or a salt or solvate thereof using compound 2

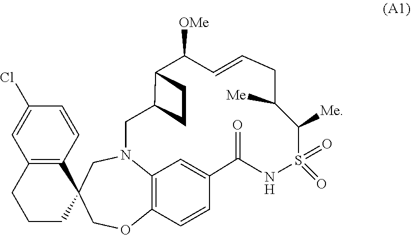

In some embodiments, the processes further include synthesizing compound A2 or a salt or solvate thereof using compound 2

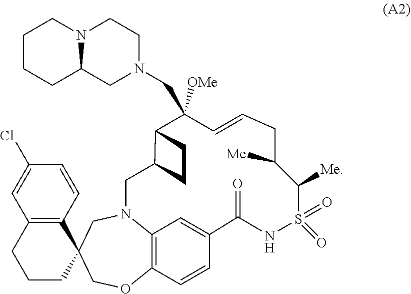

The invention is further described by reference to the following examples, which are intended to exemplify the claimed invention but not to limit it in any way.

### Examples

Unless otherwise noted, all materials were obtained from commercial suppliers and were used without further purification. Anhydrous solvents were obtained from Sigma-Aldrich (Milwaukee, WI) and used directly. All reactions involving air- or moisture-sensitive reagents were performed under a nitrogen or argon atmosphere. Purity was measured using Agilent 1100 Series high performance liquid chromatography (HPLC) systems with UV detection at 254 nm, 215 n, and 190 nm (System A: Agilent Zorbax Eclipse XDB-C8 4.6×150 mm, 5 micron, 5 to 100% ACN in H₂O with 0.1% TFA for 15 min at 1.5 mL/min; System B: Zorbax SB-C8, 4.6×75 mm, 10 to 90% ACN in H₂O with 0.1% formic acid for 12 min at 1.0 mL/min). Silica gel chromatography was generally performed with prepacked silica gel cartridges (Biotage or Teledyne-Isco). ¹H NMR spectra were recorded on a Bruker AV-400 (400 MHz) spectrometer or a Varian 400 MHz spectrometer at ambient temperature. All observed protons are reported as parts per million (ppm) downfield from tetramethylsilane (TMS) or another internal reference in the appropriate solvent indicated. Data are reported as follows: chemical shift, multiplicity (s=singlet, d=doublet, t=triplet, q=quartet, br=broad, m=multiplet), coupling constants, and number of protons. Low-resolution mass spectral (MS) data were determined on an Agilent 1100 Series LC-MS with UV detection at 254 nm and 215 nm and a low resonance electrospray mode (ESI).

The following Abbreviations are used to refer to various reagents and solvents:
AcCl Acetyl chloride
DAIB (Diacetoxyiodo)benzene
DIPEA Diisopropylethylamine
DMAP 4-Dimethylaminopyridine
DMSO Dimethylsulfoxide
(R)-DTBM-SEGPHOS (R)-(−)-5,5′-Bis[di(3,5-di-tert-butyl-4-methoxyphenyl)phosphino]-4,4′-bi-1,3-benzodioxole, [(4R)-(4,4′-bi-1,3-benzodioxole)-5,5′-diyl]bis[bis(3,5-di-tert-butyl-4-methoxyphenyl)phosphine]
equiv Equivalents
h Hour or hours
HPLC High Performance Liquid Chromatography
IPAc Isopropyl acetate
LC Liquid chromatography
LRNS Low Resonance Mass Spectrometry
MeOH Methanol
2-MeTHF 2-Methyltetrahydrofuran
min Minute or minutes
MS Mass spectrum
NMR Nuclear Magnetic Resonance
T3P Propanephosphonic acid anhydride
TEMPO 2,2,6,6-Tetramethylpiperidin-1-yl)oxyl or (2,2,6,6-tetramethylpiperidin-1-yl)oxidanyl
THF Tetrahydrofuran
TLC Thin Layer Chromatography
VinylBpin vinylboronic acid pinacol ester or 4,4,5,5-tetramethyl-2-vinyl-1,3,2-dioxaborolane

### Example 1. Preparation of ((1R,2R)-2-formylcyclobutyl)methyl acetate

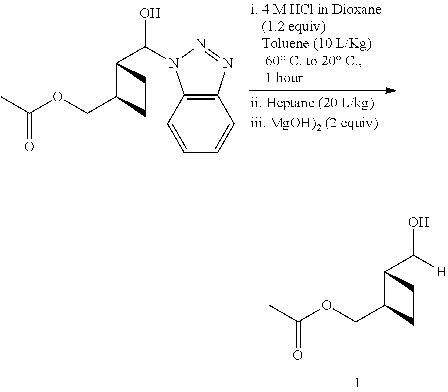 [SMILES: CC(=O)OC[C@@H]1CC[C@H]1C(O)n1nnc2ccccc21 ; CC(=O)OC[C@@H]1CC[C@H]1CO]

((1R,2R)-2-Formylcyclobutyl)methyl acetate (1). A 5 L jacketed reactor affixed with a reflux condenser and overhead stirrer was charged with ((1R,2R)-2-((1H-benzo[d][1,2,3]triazol-1-yl)(hydroxy)methyl)cyclobutyl)methyl acetate (100 g, 355 mmol, 1.00 equiv) and toluene (1.00 L, 10 L/kg) under a nitrogen atmosphere. The starting material ((1R,2R)-2-((1H-benzo[d][1,2,3]triazol-1-yl)(hydroxy)methyl)cyclobutyl)methyl acetate can be prepared using the procedure set forth in U.S. Pat. No. 9,562,061. The contents of the reactor were heated to 60° C. with constant agitation. 4 M HCl in dioxane (107 mL, 426 mmol, 1.20 equiv) was charged into the reactor. The contents of the reactor were then cooled to 20° C. Heptane (2.00 L, 20 L/kg) was charged into the reactor. The contents of the reactor were aged at 20° C. for 30 minutes before being polish-filtered into a clean 5 L jacketed reactor affixed with an overhead stirrer under a nitrogen atmosphere. Magnesium hydroxide (41.4 g, 709 mmol, 2.0 equiv) was then charged into the reactor. The contents of the reactor were aged at 20° C. for 18 hours. The contents of the reactor were then filtered and concentrated in vacuo to afford ((1R,2R)-2-formylcyclobutyl)methyl acetate (1.0) (49.6 g, 317 mmol, 88.7% yield).

Alternative Procedure for formation of (1): Triethylamine (0.30 equiv) can be used in place of magnesium hydroxide.

### Example 2. Preparation of ((1R,2R)-2-((S)-1-hydroxyallyl)cyclobutyl)methyl acetate

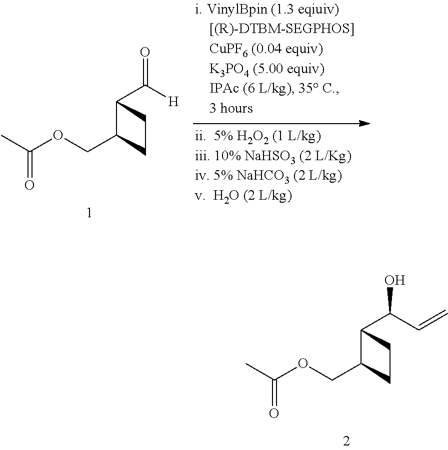

((1R,2R)-2-((S)-1-Hydroxyallyl)cyclobutyl)methyl acetate (2). A 1 L jacketed reactor affixed with a reflux condenser and overhead stirrer was charged with tetrakis(acetonitrile)copper(I) hexafluorophosphate (4.93 g, 12.8 mmol, 0.040 equiv), (R)-DTBM-SEGPHOS (commercially available)(16.7 g, 14.1 mmol, 0.044 equiv), potassium phosphate tribasic (348 g, 1.61 mol, 5.000 equiv), and isopropyl acetate (300 mL, 6 L/kg). The reactor was degassed and backfilled with nitrogen twice. The contents of the reactor were heated to 35° C. with constant agitation. At 35° C., commercially available vinylboronic acid pinacol ester (70.8 mL, 417 mmol, 1.300 equiv) was charged into the reactor. After 15 minutes, ((1R,2R)-2-formylcyclobutyl)methyl acetate (compound 1)(50.1 g, 321 mmol, 1.000 equiv) was charged into the reactor. The resulting heterogeneous mixture was agitated at 35° C. After 3 hours, the contents of the reactor were polish-filtered into a clean 1 L jacketed reactor affixed with an overhead stirrer under a nitrogen atmosphere. The product was sequentially washed with 5% H₂O₂(w/w) (50 mL, 1 L/kg), 10% NaHSO₃ (w/w) (100 mL, 2 L/kg), 5% NaHCO₃ (w/w) (100 mL, 2 L/kg), and H₂O (100 mL, 2 L/kg). The contents of the reactor were concentrated in vacuo to afford ((1R,2R)-2-((S)-1-hydroxyallyl)cyclobutyl)methyl acetate (compound 2)(45.2 g, 245 mmol, 76.4% yield).

Alternative Procedure for Preparation of ((1R,2R)-2-((S)-1-Hydroxyallyl)cyclobutyl)methyl acetate (2). A 100 mL jacketed reactor was charged with isopropyl acetate (32 mL) and potassium phosphate tribasic (55 g, 254 mmol). The mixture was agitated and the jacket temperature was set at 20° C. A catalyst stock solution was prepared by dissolving tetrakis(acetonitrile)copper(I) hexafluorophosphate (0.8 g, 2 mmol) and (R)-DTBM-SEGPHOS (commercially available)(2.6 g, 2.2 mmol) in isopropyl acetate (8 mL). The catalyst solution was then agitated at 20° C. until dissolution was observed. VinylBpin (11.5 mL, 65.8 mmol) was then added to the jacketed reactor. The jacketed reactor was then degassed and backfilled with nitrogen. The contents of the reactor were heated to 35° C. (jacket temperature) over 10 min with constant agitation. The prepared catalyst solution was then charged into the reactor followed by a 2 mL rinse of the vial. The mixture was then aged for 3 minutes after the addition. ((1R,2R)-2-Formylcyclobutyl)methyl acetate (compound 1)(13.4 g, 51.5 mmol) was then charged into the reactor via syringe pump over 60 minutes. The resulting heterogeneous mixture was agitated at 35° C. After 99.5% conversion to desired product was observed, the contents of the reactor were polish-filtered into a clean 250 mL round bottom flask. The reactor and filter cake were washed with isopropyl acetate (4×16 mL).

Procedure for oxidative workup and crystallization of the DTBM-SEGPHOS ligand. The black reaction liquor of a batch produced on a different scale as that described above was charged into a 2 L reactor. An aqueous solution of H₂O₂(5%, 2 L/kg, 200 mL) was charged into the reactor over 30 minutes. The biphasic mixture was then agitated at ambient temperature for one hour. An aqueous solution of NaHSO₃ (10%, 3 L/kg, 300 mL) was then charged into the reactor over 30 minutes. Agitation was then stopped, and the green aqueous layer was drained in a Schott bottle (pH=2). A solution of NaHCO₃ (5%, 4 L/kg, 400 mL) was then charged into the reactor and some outgassing was observed. The resulting biphasic mixture was then agitated at ambient temperature for one hour. Next, agitation was stopped and a fast phase split was observed giving a colorless aqueous layer and a pale yellow organic layer (pH=8). Water (2 L/kg, 200 mL) was then charged into the reactor, and the mixture was agitated for 15 minutes. Next, agitation was stopped and a fast phase split was observed giving a colorless aqueous phase and a pale yellow organic layer (pH=7). The organic layer was drained into a 3 L round bottom flask and concentrated in vacuo. The resulting residue was taken up in MeOH (300 mL, 3 L/kg) and then concentrated in vacuo. The resulting residue was taken up again in MeOH (300 mL) and then concentrated in vacuo. The resulting residue was transferred to a clean 1 L reactor and diluted with MeOH (300 mL, 3 L/kg) and stirred at 20° C. Water (100 mL, 1 L/kg) was then charged slowly into the reactor and a seed crystal of DTBM-SEGPHOS-Oxide (500 mg) was charged into the reactor. The resulting slurry was aged overnight to relieve supersaturation. The resulting mixture was polish-filtered through a medium porosity frit into a 2 L round bottom flask. The vessel and the cake were washed with a 25% aqueous solution of MeOH (100 mL, 1 L/kg). The resulting solution was diluted with toluene (500 mL, 5 L/kg) and then the solution was concentrated in vacuo. Three toluene charges of 5 L/kg each followed by distillation after each charge were used to remove all the MeOH and water from the reaction stream.

The following scheme details how treatment of the reaction mixture with hydrogen peroxide oxidizes the phosphines in the ligand to phosphine oxides. The diphosphine oxide readily crystallizes away from the mixture allowing purification and recovery of the ligand as the diphosphine oxide and concomitantly providing purified compound 2. Reduction of the phosphine oxides provides the (R)-DTBM-SEGPHOS ligand which can be reused reducing the costs of the ligand. Various reducing agents such as HSiCl₃ and others useful for reducing phosphine oxides may be used to convert the phosphine oxides back to the phosphines.

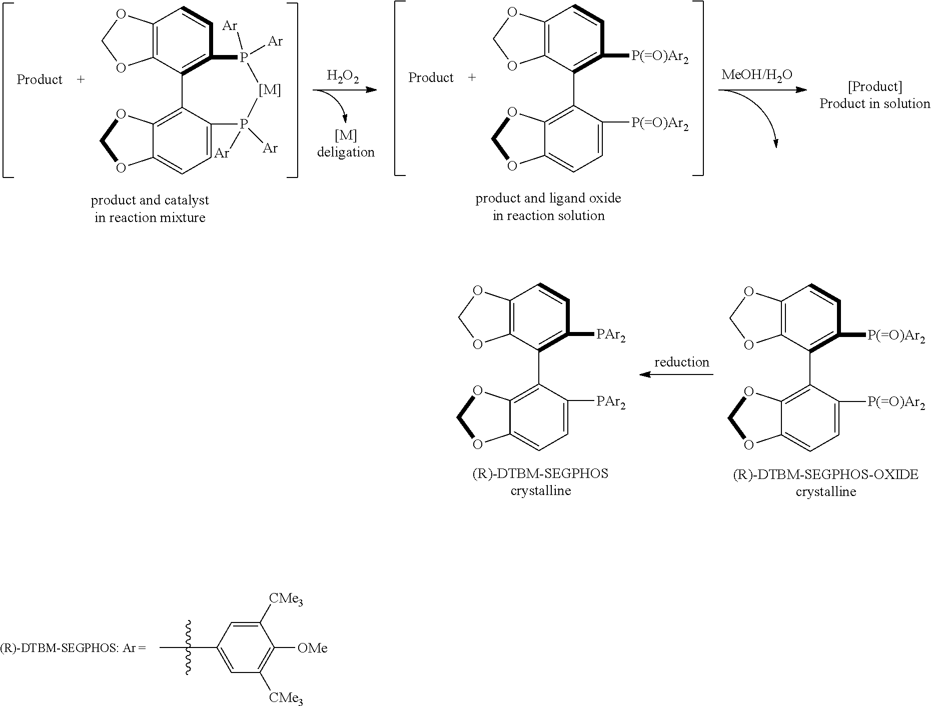

Reduction of Phosphine Oxides

To a 100 mL glass pressure reactor equipped with a stir bar was charged (R)-DTBM-SEGPHOS-OXIDE (1.0 g, 1.0 eq, 0.83 mmol) followed by toluene (10 mL). Trichlorosilane (1.1 mL, 13.0 eq, 11 mmol) was added to the reaction followed by triethylamine (1.9 mL, 16 eq, 13 mmol) and the reaction was allowed to stir sealed at 110° C. overnight. After cooling, TLC analysis (20% DCM in heptane) showed conversion to a new spot. The reaction was worked up by addition of deionized water (15 mL) and extraction with ethyl acetate (30 mL, 3 times). The combined organic layers were dried over Na₂SO₄, filtered and concentrated to 0.91 g (93.8%) glassy foam.

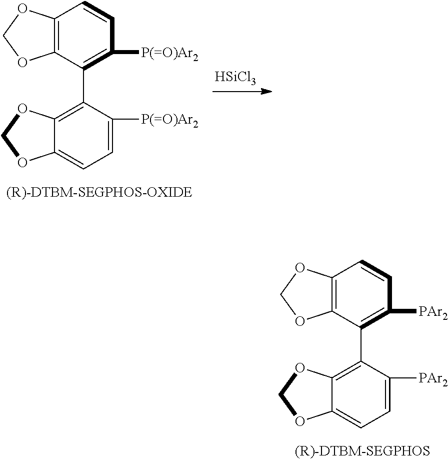

### Example 3. Preparation of (S)-1-((1R,2R)-2-(acetoxymethyl)cyclobutyl)allyl 4-bromobenzoate

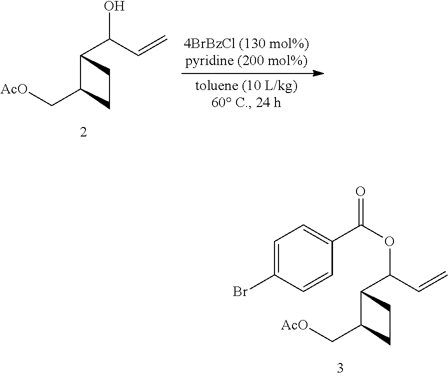 [SMILES: C=CC(O)[C@@H]1CC[C@H]1COC(C)=O ; C=CC(OC(=O)c1ccc(Br)cc1)[C@@H]1CC[C@H]1COC(C)=O]

(S)-1-((1R,2R)-2-(Acetoxymethyl)cyclobutyl)allyl 4-bromobenzoate (3). A 3100 L glass-lined reactor was flushed with nitrogen and charged with compound 2 (363.5 kg, 25.3 w/w % in toluene, 1.00 equiv.), pyridine (81 L, 2.0 equiv.), and toluene (287 L, 3.1 L/kg). The mixture was stirred at 20° C. until homogenous. A solution of commercially available 4-bromobenzoyl chloride (143 kg, 1.30 equiv.) in toluene (380 L, 4.1 L/kg) was then charged into the reaction mixture. The reaction mixture was heated to 60° C. and held for 4 hours or until the reaction was judged complete by HPLC analysis. The reaction was cooled to 5° C. and then quenched with 1M HCl (367 L, 4 L/kg). The reaction mixture was filtered through a 20 μm filter into a clean 14,300 L glass-lined reactor forward rinsing with toluene (184 L, 2 L/kg). The biphasic mixture was warmed to 20° C. and the phases were split. The toluene solution was sequentially washed with sodium bicarbonate solution (5 w/w %, 368 L, 4 L/kg) and water (368 L, 4 L/kg). The toluene solution was then concentrated to a volume of ˜184 L, maintaining the internal temperature <40° C. n-Heptane (460 L, 5 L/kg) was then charged into the reactor, and the resulting solution was cooled to 5° C. After stirring for 1 hour at 5° C., the reaction mixture was filtered through a 0.5 μm sparkler filter into a clean 6700 L glass-lined reactor forward rinsing with n-heptane (110 L, 1.2 L/kg). The mixture was then concentrated to a volume of ˜262 L while maintaining the internal temperature <40° C. The resulting solution of (S)-1-((1R,2R)-2-(acetoxymethyl)cyclobutyl)allyl 4-bromobenzoate (compound 3) was cooled to 20° C. and used directly into the next step. ¹H NMR (600 MHz, DMSO) δ 7.92 (d, J=8.5 Hz, 2H), 7.76 (d, J=8.5 Hz, 2H), 5.85 (ddd, J=17.1, 10.6, 6.1 Hz, 1H), 5.42 (ddt, J=8.0, 6.1, 1.4 Hz, 1H), 5.27 (dt, J=17.1, 1.4 Hz, 1H), 5.20 (dt, J=10.6, 1.4 Hz, 1H), 3.97 (dd, J=11.4, 6.0 Hz, 1H), 3.95 (dd, J=11.4, 6.0 Hz, 1H), 2.55-2.49 (m, 1H), 2.47 (qui, J=8.0 Hz, 1H), 1.94-1.87 (m, 2H), 1.87 (s, 3H), 1.72 (dq, J=10.7, 9.1 Hz, 1H), 1.64 (dq, J=11.3, 9.1 Hz, 1H). ¹³C NMR (151 MHz, DMSO) δ 170.3, 164.3, 134.5, 131.9, 131.1, 128.9, 127.5, 117.1, 77.6, 66.6, 40.5, 36.4, 20.5, 20.5, 19.9. LRMS (ESI): Calculated for C₇H₁₉BrO₄+H: 367, found: 367.

### Example 4. Preparation of (S)-1-((1R,2R)-2-(hydroxymethyl)cyclobutyl)allyl 4-bromobenzoate

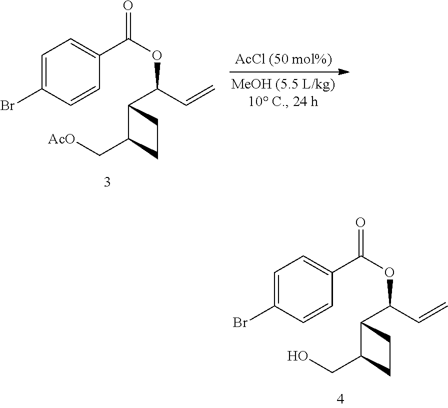 [SMILES: C=C[C@H](OC(=O)c1ccc(Br)cc1)[C@@H]1CC[C@H]1COC(C)=O ; C=C[C@H](OC(=O)c1ccc(Br)cc1)[C@@H]1CC[C@H]1CO]

(S)-1-((1R,2R)-2-(Hydroxymethyl)cyclobutyl)allyl 4-bromobenzoate (4). A 6700 L glass-lined reactor containing a ˜262 L solution of compound 3 was charged with methanol (938 L, 5.5 L/kg) and cooled to 1° C. Commercially available acetyl chloride (16 L, 0.5 equiv.) was charged into the reactor at a rate to maintain the internal temperature <5° C. The reaction mixture was then stirred at 10° C. for 10 hours or until judged complete by HPLC analysis. The reaction mixture was diluted with toluene (1750 L, 10 L/kg) before being quenched with sodium bicarbonate solution (5 w/w %, 852 L, 5 L/kg) and sodium chloride solution (5 w/w %, 170 L, 1 L/kg). The biphasic mixture was warmed to 20° C. and the phases were separated. The toluene layer was washed with water (852 L, 5 L/kg). The mixture was then concentrated to a volume of ˜186 L while maintaining the internal temperature <40° C. (S)-1-((1R,2R)-2-(Hydroxymethyl)cyclobutyl)allyl 4-bromobenzoate (compound 4) (317.5 kg, 48.8 w/w % in toluene) was obtained by HPLC assay and used directly in the next step. ¹H NMR (600 MHz, DMSO) δ 7.91 (d, J=8.6 Hz, 2H), 7.76 (d, J=8.6 Hz, 2H), 5.86 (ddd, J=17.2, 10.7, 5.7 Hz, 1H), 5.40 (ddt, J=8.0, 5.7, 1.4 Hz, 1H), 5.26 (dt, J=17.2, 1.4 Hz, 1H), 5.19 (dt, J=10.7, 1.4 Hz, 1H), 4.43 (t, J=5.7 Hz, 1H), 3.36 (dt, J=10.3, 5.7 Hz, 1H), 3.31 (dt, J=10.3, 5.7 Hz, 1H), 2.42 (qui, J=8.0 Hz, 1H), 2.30 (quid, J=8.0, 4.2 Hz, 1H), 1.90-1.77 (m, 2H), 1.73-1.60 (m, 2H). ¹³C NMR (151 MHz, DMSO) δ 164.4, 134.8, 131.9, 131.1, 129.1, 127.4, 116.9, 78.1, 64.0, 40.0, 39.6, 20.4, 19.9. LRMS (ESI): Calculated for C₁₅H₁₇BrO₃+H: 325, found: 325.

### Example 5. Preparation of (S)-1-((1R,2R)-2-formylcyclobutyl)allyl 4-bromobenzoate

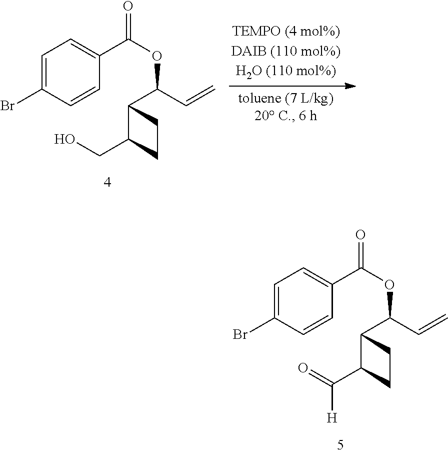 [SMILES: C=C[C@H](OC(=O)c1ccc(Br)cc1)[C@@H]1CC[C@H]1CO ; C=C[C@H](OC(=O)c1ccc(Br)cc1)[C@@H]1CC[C@H]1C=O]

(S)-1-((1R,2R)-2-Formylcyclobutyl)allyl 4-bromobenzoate (5). A 3600 L stainless steel reactor was flushed with nitrogen and charged with compound 4 (317.5 kg, 48.8 w/w % in toluene, 1.00 equiv.) and toluene (930 L, 6 L/kg). The mixture was stirred at 20° C. until homogenous. Water (9.5 L, 1.10 equiv.) and commercially available (diacetoxyiodo)benzene (169 kg, 1.10 equiv.) were charged into the reactor. The heterogeneous mixture was cooled to 15° C. A solution of commercially available TEMPO (2.9 kg, 0.04 equiv.) in toluene (155 L, 1 L/kg) was charged into the reactor at a rate to maintain the internal temperature <20° C. The reaction mixture was then warmed to 20° C. and held for 12 hours or until the reaction was judged complete by HPLC analysis. The reaction was quenched with sodium thiosulfate solution (5 w/w %, 775 L, 5.0 L/kg) and the phases were separated. The toluene layer was sequentially washed with sodium carbonate solution (5 w/w %, 775 L, 5.0 L/kg) and twice with water (775 L, 5.0 L/kg). The mixture was concentrated to a volume of ˜465 L, maintaining the internal temperature <40° C. The resulting solution of (S)-1-((1R,2R)-2-formylcyclobutyl)allyl 4-bromobenzoate (compound 5) was cooled to 20° C. and used directly in the next step. ¹H NMR (600 MHz, DMSO) δ 9.61 (d, 1.9 Hz, 1H), 7.90 (d, 8.2 Hz, 2H), 7.75 (d, 8.2 Hz, 2H), 5.85 (dddd, 17.3,10.6,6.0,0.6 Hz, 1H), 5.44 (ddt, 7.4,6.0,1.4 Hz, 1H), 5.30 (dtd, 17.3,1.4,0.6 Hz, 1H), 5.22 (dq, 10.6,1.4,0.6 Hz, 1H), 3.23-3.15 (m, 1H), 2.93-2.85 (m, 1H), 2.11-2.02 (m, 1H), 2.00-1.94 (m, 1H), 1.89-1.82 (m, 2H); ¹³C NMR (151 MHz, DMSO) δ 202.2, 164.3, 134.1, 131.9, 131.1, 128.8, 127.5, 117.6, 77.2, 47.3, 38.4, 19.9, 18.0. LRMS (ESI): Calculated for C₁₅H₁₅BrO₃+H: 323, found: 323.

### Example 6. Preparation of (1S)-1-((1R,2R)-2-((1H-Benzo[d][1,2,3]triazol-1-yl)(hydroxy)methyl)cyclobutyl)allyl 4-bromobenzoate

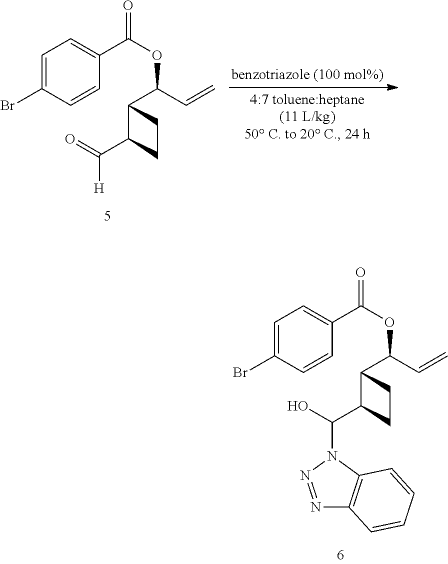 [SMILES: C=C[C@H](OC(=O)c1ccc(Br)cc1)[C@@H]1CC[C@H]1C=O ; C=C[C@H](OC(=O)c1ccc(Br)cc1)[C@@H]1CC[C@H]1C(O)n1nnc2ccccc21]

(1S)-1-((1R,2R)-2-((1H-Benzo[d][1,2,3]triazol-1-yl)(hydroxy)methyl)cyclobutyl)allyl 4-bromobenzoate (6). A 3600 L stainless steel reactor containing a ˜465 L solution of 5 was charged with benzotriazole (56.5 kg, 1.00 equiv.). The mixture was stirred at 20° C. until homogeneous. The resulting solution was filtered through a 0.5 μm polyester filter into a clean 3600 L stainless steel reactor forward rinsing with toluene (155 L, 1 L/kg.). The reaction mixture was then heated to 50° C. Next, n-Heptane (310 L, 2 L/kg.) was charged into the reactor at a rate to maintain the internal temperature >45° C. Milled compound 6 seed (3.2 kg, 2.0 w/w %) was charged into the reactor and the suspension was held at 50° C. for 1 hour. n-Heptane (622.5 L, 4 L/kg) was dosed into the reactor over 10 hours maintaining the internal temperature at 50° C. before starting a cooling ramp to 20° C. over 4 hours. n-Heptane (310 L, 2 L/kg) was then added to the reactor over 2 hours maintaining the internal temperature at 20° C. before initiating a 4 hour hold. The heterogeneous mixture was transferred into a 1260 L Hastelloy agitated filter dryer and deliquored. The cake was sequentially washed with a 1:1 mixture of toluene:n-heptane (310 L, 2 L/kg) and n-heptane (310 L, 2 L/kg). The cake was dried under vacuum maintaining the internal temperature <50° C. (1$-1-((1R,2R)-2-((1H-Benzo[d][1,2,3]triazol-1-yl)(hydroxy)methyl)cyclobutyl)allyl 4-bromobenzoate (compound 6) (170 kg) was isolated by HPLC assay in an 80% molar yield. ¹H NMR (600 MHz, DMSO) δ 8.01 (dt, J=8.3, 1.0 Hz, 1H), 7.92 (d, J=8.6 Hz, 2H), 7.88 (dt, J=8.3, 1.0 Hz, 1H), 7.73 (d, J=8.6 Hz, 2H), 7.53 (ddd, J=8.3, 6.9, 1.0 Hz, 1H), 7.39 (ddd, J=8.3, 6.9, 1.0 Hz, 1H), 7.22 (d, J=5.8 Hz, 1H), 6.29 (dd, J=8.7, 5.8 Hz, 1H), 5.96 (ddd, J=17.3, 10.6, 6.4 Hz, 1H), 5.57 (tt, J=6.4, 1.2 Hz, 1H), 5.33 (dt, J=17.3, 1.4 Hz, 1H), 5.25 (dt, J=10.6, 1.4 Hz, 1H), 3.20 (qui, J=8.7 Hz, 1H), 2.78 (qui, J=8.7 Hz, 1H), 1.93 (dtd, J=11.6, 8.7, 3.8 Hz, 1H), 1.75 (dq, J=11.6, 8.7 Hz, 1H), 1.62 (ddd, J=11.9, 8.7, 3.8 Hz, 1H), 1.56 (dt, J=11.9, 8.7 Hz, 1H); ¹³C NMR (150 MHz, DMSO) δ 164.6, 145.5, 134.3, 131.7, 131.7, 131.2, 129.4, 127.1, 127.0, 123.9, 119.1, 117.7, 111.6, 85.9, 77.4, 41.7, 40.6, 19.4, 19.0. LRMS (ESI): Calculated for C₂₁H₂₀BrN₃O₃+H: 442, found: 442.

### Example 7. Preparation of (S)-5-(((1R,2R)-2-((S)-1-((4-bromobenzoyl)oxy)allyl)cyclobutyl)methyl)-6′-chloro-3′,4,4′,5-tetrahydro-2H,2′H-spiro[benzo[b][1,4]oxazepine-3,1′-naphthalene]-7-carboxylic acid

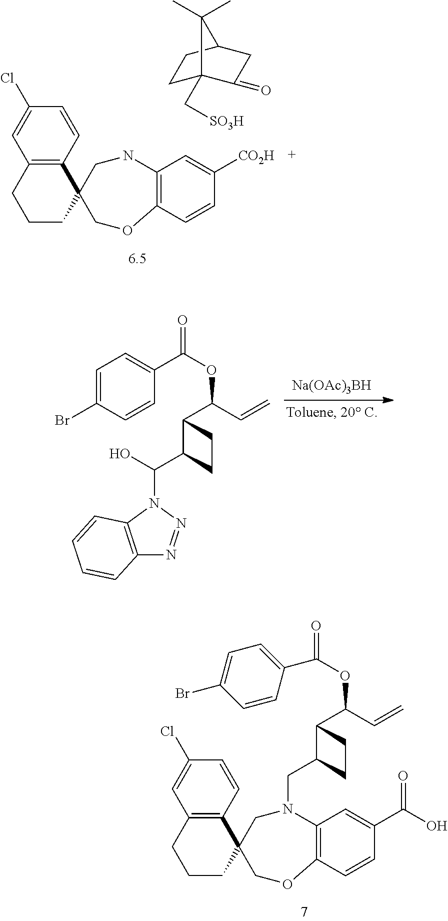

(S)-5-(((1R,2R)-2-((S)-1-((4-Bromobenzoyl)oxy)allyl)cyclobutyl)methyl)-6′-chloro-3′,4,4′,5-tetrahydro-2H,2′H-spiro[benzo[b][1,4]oxazepine-3,1′-naphthalene]-7-carboxylic acid (7). To a 500 mL, glass lined, jacketed reactor was charged 25.0 g of 6.5 (1.0 equiv, 43.4 mmol), followed by 21.9 g of 6 (1.1 equiv, 47.7 mmol, 70.4 wt %) and 250 mL of toluene (10 L/kg). Compound 6.5 may be prepared as described in U.S. Pat. No. 9,562,061. The resultant slurry mixture was stirred at 20° C. for 30 min. To the reactor was then charged NaBH(OAc)₃ (11.5 g, 1.25 equiv) in 0.25 equivalent portions at 20° C., with each portion charged at least 15 minutes apart. The reaction was stirred at 20° C. for >5 hours, until LC analysis confirmed complete consumption of compound 6.5. To the reaction mixture was then slowly charged an aqueous solution of NaCl and NaHCO₃ to control gas evolution. The batch was stirred at 20° C. for >30 minutes. The aqueous phase was then removed after phase separation. To the reactor containing the organic phase was charged aqueous H₃PO₄, and the resulting mixture was stirred at 20° C. for >15 minutes. The aqueous phase was removed after phase separation. The aqueous H₃PO₄ washing sequence was repeated two more times. To the reactor containing the organic phase was then charged aqueous NaCl, and the mixture was stirred at 20° C. for >15 minutes. The aqueous phase was removed after phase separation. The batch was then concentrated under reduced pressure at ≤55° C. The batch was then cooled to 20° C. Next, (S)-5-(((1R,2R)-2-((S)-1-((4-bromobenzoyl)oxy)allyl)cyclobutyl)methyl)-6′-chloro-3′,4,4′,5-tetrahydro-2H,2′H-spiro[benzo[b][1,4]oxazepine-3,1′-naphthalene]-7-carboxylic acid (compound 7) seed crystals were added to induce crystallization, and the slurry was held at 20° C. for >1 hour. Heptane was then charged to the reactor. After addition, the suspension was stirred at 20° C. for >1 hour. (S)-5-(((1R,2R)-2-((S)-1-((4-Bromobenzoyl)oxy)allyl)cyclobutyl)methyl)-6′-chloro-3′,4,4′,5-tetrahydro-2H,2′H-spiro[benzo[b][1,4]oxazepine-3,1′-naphthalene]-7-carboxylic acid (compound 7) was obtained after filtration and washing with 2/1 heptane/toluene and dried at 40° C. under vacuum. Compound 7 was obtained in 85.5 wt %, 85.0% isolated yield over the two steps. ¹H NMR (600 MHz, CDCl₃) δ 7.86 (d, J=8.6 Hz, 2H), 7.64 (d, J=8.5 Hz, 1H), 7.50 (d, J=8.6 Hz, 2H), 7.47 (dd, J=8.2, 1.9 Hz, 1H), 7.44 (d, J=1.9 Hz, 1H), 7.16 (dd, J=8.5, 2.3 Hz, 1H), 7.08 (d, J=2.3 Hz, 1H), 6.93 (d, J=8.2 Hz, 1H), 5.84 (ddd, J=17.1, 10.6, 6.4 Hz, 1H), 5.49 (bt, J=6.4 Hz, 1H), 5.36 (dt, J=17.1, 1.2 Hz, 1H), 5.22 (dt, J=10.6, 1.2 Hz, 1H), 4.12 (d, J=12.1 Hz, 1H), 4.08 (d, J=12.1 Hz, 1H), 3.59 (dd, J=14.8, 4.1 Hz, 1H), 3.52 (d, J=14.4 Hz, 1H), 3.35 (dd, J=14.8, 9.0 Hz, 2H), 3.32 (d, J=14.4 Hz, 1H), 2.78-2.75 (m, 1H), 2.75-2.71 (m, 2H), 2.47 (qui, J=8.5 Hz, 1H), 2.12-2.02 (m, 1H), 2.00-1.92 (m, 1H), 1.93-1.85 (m, 2H), 1.85-1.77 (m, 1H), 1.78-1.69 (m, 2H), 1.56 (bt, J=11.0 Hz, 1H); ¹³C NMR (151 MHz, CDCl₃) δ 171.8, 165.1, 153.7, 141.0, 139.0, 138.8, 134.3, 132.1, 131.7, 131.0, 129.5, 129.1, 128.6, 128.1, 126.6, 123.7, 121.7, 120.8, 117.5, 117.0, 79.4, 78.0, 60.9, 58.8, 43.0, 41.8, 36.2, 30.2, 29.0, 25.9, 21.2, 19.0. LRMS (ESI): Calculated: 650.1; Found: 650.

### Example 8. Preparation of ((S)-5-(((1R,2R)-2-((S)-1-((4-bromobenzoyl)oxy)allyl)cyclobutyl)methyl)-6′-chloro-3′,4,4′,5-tetrahydro-2H,2′H-spiro[benzo[b][1,4]oxazepine-3,1′-naphthalene]-7-carbonyl)(((2R,3S)-3-methylhex-5-en-2-yl)sulfonyl)amide piperazine salt

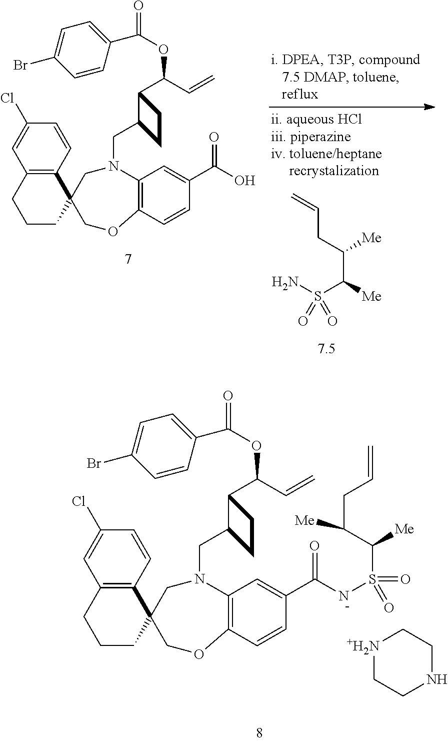

((S)-5-(((1R,2R)-2-((S)-1-((4-Bromobenzoyl)oxy)allyl)cyclobutyl)methyl)-6′-chloro-3′,4,4′,5-tetrahydro-2H,2′H-spiro[benzo[b][1,4]oxazepine-3,1′-naphthalene]-7-carbonyl)(((2R,3S)-3-methylhex-5-en-2-yl)sulfonyl)amide piperazine salt (8). A flask was charged with compound 7 (10 g, 85 wt %, 13.2 mmol)), toluene (50 mL) and DIPEA (6.0 mL, 3.5 equiv.). To the homogenous solution was added 50 wt % T3P in toluene (13.6 mL, 1.5 equiv.), compound 7.5 (2.6 g, 1.1 equiv.) and DMAP (1.6 g, 1.0 equiv.). Compound 7.5 may be prepared as described in U.S. Pat. No. 9,562,061. The resulting mixture was then heated at reflux overnight. Next, the reaction was cooled to room temperature and quenched with 1M aqueous HCl (50 mL). The aqueous layer was separated, and the organic layer was washed twice with 1M aq HCl (50 mL) and once with water (50 mL). The organic layer was polish-filtered, washed with toluene (50 mL) and concentrated to approximately 50 mL. Piperazine (1.14 g, 1.0 equiv.) was charged into the toluene solution and the mixture was stirred at 60° C. for 1 hour. The solution was cooled to room temperature and compound 8 piperazine salt seed crystals were charged into the mixture. The slurry was then stirred and heptane (22 mL) was charged into the mixture. Upon complete addition, the slurry was warmed to 50° C. and additional heptane (21 mL) was charged into the mixture. The slurry was cooled to room temperature and filtered, and the cake was washed twice with 1:1 toluene/heptane (50 mL) and dried to deliver ((S)-5-(((1R,2R)-2-((S)-1-((4-Bromobenzoyl)oxy)allyl)cyclobutyl)methyl)-6′-chloro-3′,4,4′,5-tetrahydro-2H,2′H-spiro[benzo[b][1,4]oxazepine-3,1′-naphthalene]-7-carbonyl)(((2R,3S)-3-methylhex-5-en-2-yl)sulfonyl)amide piperazine salt (compound 8) as an off-white crystalline solid (11.4 g, 85 wt %, 82% yield): ¹H NMR (600 MHz, DMSO-d₆): δ 7.79 (d, 8.6 Hz, 2H), 7.67 (d, 8.6 Hz, 2H), 7.53 (d, 1.9 Hz, 1H), 7.48 (d, 8.5 Hz, 1H), 7.31 (dd, 8.2,1.9 Hz, 1H), 7.14 (dd, 8.5,2.4 Hz, 1H), 7.12 (d, 2.4 Hz, 1H), 6.76 (d, 8.2 Hz, 1H), 5.86 (ddd, 17.2,10.7,6.4 Hz, 1H), 5.71 (ddt, 17.1,10.2,7.0 Hz, 1H), 5.41 (bt, 6.4 Hz, 1H), 5.27 (dt, 17.2,1.4 Hz, 1H), 5.15 (dt, 10.7,1.4 Hz, 1H), 5.00 (dq, 17.1,1.5 Hz, 1H), 4.95 (ddt, 10.2,2.4,1.5 Hz, 1H), 3.95 (d, 12.0 Hz, 1H), 3.87 (d, 12.0 Hz, 1H), 3.38 (dd, 14.2,8.0 Hz, 1H), 3.37 (qd, 7.1,2.6 Hz, 1H), 3.30 (dd, 14.2,5.5 Hz, 1H), 3.20 (d, 14.1 Hz, 1H), 3.15 (d, 14.1 Hz, 1H), 2.90 (s, 8H), 2.66 (bt, 6.4 Hz, 2H), 2.59 (td, 8.0,5.5 Hz, 1H), 2.49 (qui, 8.0 Hz, 1H), 2.34 (sxtd, 7.0,2.6 Hz, 1H), 1.97 (m, 3H), 1.85 (m, 2H), 1.73 (m, 2H), 1.66 (m, 2H), 1.55 (ddd, 13.5,9.8,4.0 Hz, 1H), 1.08 (d, 7.1 Hz, 3H), 0.94 (d, 7.0 Hz, 3H); ¹³C NMR (150 MHz, DMOS-d₆): δ 169.8, 164.4, 150.9, 140.7, 139.6, 138.8, 137.3, 134.6, 134.4, 131.9, 131.0, 130.7, 129.4, 128.8, 128.2, 127.3, 125.9, 119.8, 119.5, 117.2, 116.4, 116.0, 78.7, 77.6, 61.2, 58.2, 57.2, 43.2, 42.3, 41.4, 40.0, 35.8, 31.4, 29.6, 28.5, 24.2, 20.2, 18.2, 14.5, 8.4; LRMS (ESI): Calcd. for C₄₁H₄₆BrClN₂O₆S+Na: 831.2, found: 831.2.

### Example 9. Preparation of (1S,11′R,12′S,16′S,16a′R,18a′R,E)-16′-((4-bromobenzyl)oxy)-6-chloro-11′,12′-dimethyl-3,4,12′,13′,16′,16a′,17′,18′,18a′,19′-decahydro-1′H,2H,3′H,11′H-spiro[naphthalene-1,2′-[5,7]ethenocyclobuta[i][1,4]oxazepino[3,4-f][1]thia[2,7]diazacyclohexadecin]-8′(9′H)-one 10′,10′-dioxide

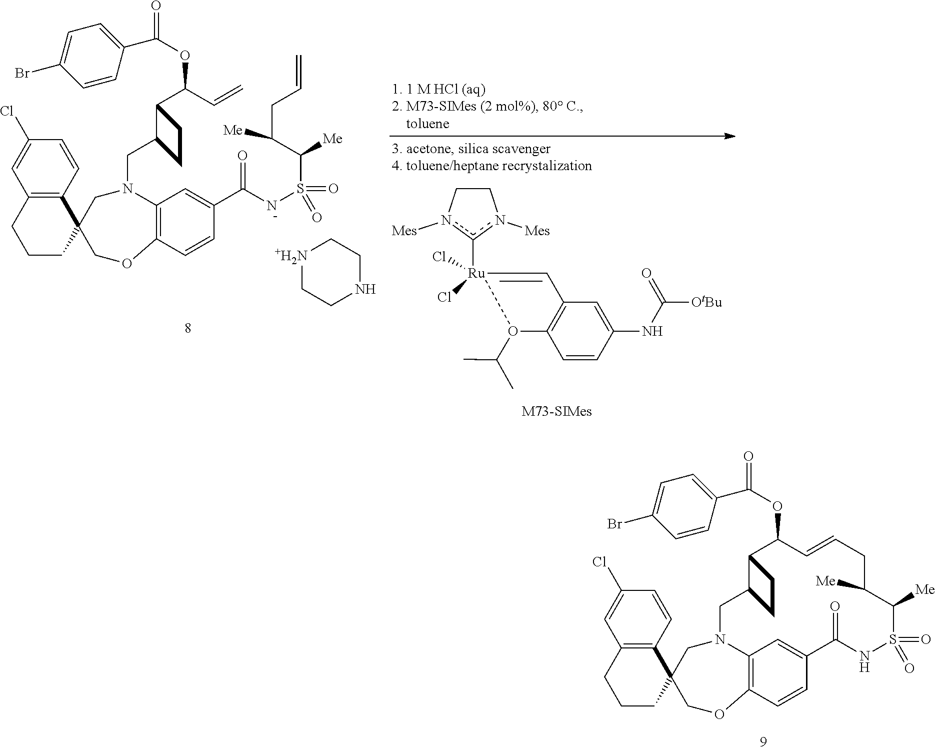

(1S,11′R,12′S,16′S,16a′R,18a′R,E)-16′-((4-Bromobenzyl)oxy)-6-chloro-11′,12′-dimethyl-3,4,12′,13′,16′,16a′,17′,18′,18a′,19′-decahydro-1′H,2H,3′H,11′H-spiro[naphthalene-1,2′-[5,7]ethenocyclobuta[i][1,4]oxazepino[3,4-f][1]thia[2,7]diazacyclohexadecin]-8′(9′H)-one 10′,10′-dioxide (9). In a jacketed vessel, compound 8 piperazine salt (70 g) was stirred in toluene (1.4 L, 20 L/kg) in the presence of an aqueous HCl 1N solution (0.35 L) at room temperature for 1 hour. Upon separation of the layers, the organic layer was washed two more times with HCl 1 N (2×0.35 L, 10 L/kg) for complete removal of residual piperazine. The resulting organic layer was washed twice with deionized water (2×0.35 L, 10 L/kg). The organic layer with the free form of compound 8 was then concentrated under vacuum until 700 mL was reached. In a second vessel, a solution of catalyst M73-SIMes (1.287 g, 1.734 mmol, 0.022 equiv.) was prepared in a dichloromethane (0.35 L, 5 g/mL) and toluene mixture (0.35 L, 5 g/mL). Toluene (2.80 L, 40 L/kg) was charged into a third large vessel equipped with a condenser, and the mixture was heated to 75-85° C. (target 80° C.). A controlled vacuum was set to an internal pressure of 300-500 torr was then applied. The catalyst solution and the toluenic compound 8 solution were simultaneously charged over 60-90 minutes to the vessel containing toluene at 80° C. under 300-500 torr pressure. After addition was complete, the solution was stirred for 1 hour before sampling for conversion. Upon completion of the reaction (monitored by LC), the batch was pressurized to 1 atm with a flow of nitrogen and cooled down to 45° C. Commercially available diethyleneglycol monovinylether (256 uL, 1.874 mmol, 0.024 equiv.) was added to quench the remaining active catalyst. After 1 hour, the batch was distilled under vacuum to approximately 700 mL of toluene. The mixture was then cooled to room temperature and diluted with acetone (0.7 L, 10 L/kg) to reach a 1:1 toluene/acetone solution. Silia-MetS-Thiol scavenger (35.0 g) was then charged into the mixture, and the slurry was warmed to 50° C. with agitation to scavenge ruthenium metal. After 16 hours of stirring, the batch was filtered and the spent silica was washed twice with 1:1 toluene/acetone (2×0.63 L, 18 L/kg). Filtrate and washes were combined and concentrated under vacuum to reduce the total volume to approximately 700 mL. The batch was then held at 45° C. during 2 hours to induce self-seeding. Heptane (0.28 L, 4 L/kg) was dosed into the slurry at 45° C. over 3 hours, followed by a progressive cool down to 20-25° C. The slurry was filtered under vacuum, and the cake was washed twice with 2:1 toluene:heptane (2×0.21 L, 6 L/kg). The solid was then dried under vacuum at 40° C. to provide (1S,11′R,12′S,16′S,16a′R,18a′R,E)-16′-((4-bromobenzyl)oxy)-6-chloro-11′,12′-dimethyl-3,4,12′,13′,16′,16a′,17′,18′,18a′,19′-decahydro-1′H,2H,3′H,11′H-spiro[naphthalene-1,2′-[5,7]ethenocyclobuta[i][1,4]oxazepino[3,4-f][1]thia[2,7]diazacyclohexadecin]-8′(9′H)-one 10′,10′-dioxide (compound 9) as a white solid (48.9 g, 80-85% yield). ¹H NMR (600 MHz, CDCl₃) δ 8.46 (s, 1H), 7.71 (d, J=8.6 Hz, 2H), 7.56 (d, J=8.5 Hz, 2H), 7.54 (d, J=8.7 Hz, 1H), 7.16 (dd, J=8.7, 2.3 Hz, 1H), 7.10 (d, J=2.0 Hz, 1H), 7.07 (d, J=2.3 Hz, 1H), 7.01 (dd, J=8.1, 2.0 Hz, 1H), 6.95 (d, J=8.1 Hz, 1H), 5.97 (ddd, J=15.2, 9.1, 4.4 Hz, 1H), 5.73 (ddt, J=15.2, 8.2, 1.4 Hz, 1H), 5.59 (dd, J=8.2, 4.8 Hz, 1H), 4.30 (qd, J=7.3, 1.2 Hz, 1H), 4.08 (d, J=12.4 Hz, 1H), 4.06 (d, J=12.4 Hz, 1H), 3.97 (dd, J=15.5, 3.2 Hz, 1H), 3.57 (d, J=14.4 Hz, 1H), 3.17 (d, J=14.4 Hz, 1H), 3.03 (dd, J=15.5, 9.1 Hz, 1H), 2.81-2.76 (m, 1H), 2.77-2.72 (m, 1H), 2.67 (qd, J=9.2, 4.7 Hz, 1H), 2.46 (quid, J=9.1, 3.2 Hz, 1H), 2.14-2.04 (m, 3H), 2.04-1.99 (m, 2H), 2.00-1.88 (m, 3H), 1.85-1.74 (m, 1H), 1.69 (dq, J=10.6, 9.1 Hz, 1H), 1.45 (d, J=7.3 Hz, 3H), 1.38 (tt, J=13.2, 2.3 Hz, 1H), 1.02 (d, J=6.7 Hz, 3H). ¹³C NMR (151 MHz, CDCl₃) δ 166.5, 164.8, 152.8, 140.8, 139.3, 138.7, 134.2, 132.2, 131.7, 131.1, 129.6, 129.4, 128.5, 127.9, 126.7, 126.4, 126.3, 120.9, 116.0, 115.7, 80.2, 75.9, 59.4, 58.1, 57.8, 41.7, 37.5, 33.7, 33.4, 30.1, 28.2, 26.6, 19.8, 19.0, 15.4, 5.9. LRMS (ESI): Calcd. for C₃₉H₄₂BrClN₂O₆S+Na: 803.1, found: 803.1.

### Example 10. Preparation of (1S,11′R,12′S,16′S,16a′R,18a′R,E)-6-chloro-16′-hydroxy-11′,12′-dimethyl-3,4,12′,13′,16′,16a′,17′,18′,18a′,19′-decahydro-1′H,2H,3′H,11′H-spiro[naphthalene-1,2′-[5,7]ethenocyclobuta[i][1,4]oxazepino[3,4-f][1]thia[2,7]diazacyclohexadecin]-8′(9′H)-one 10′,10′-dioxide

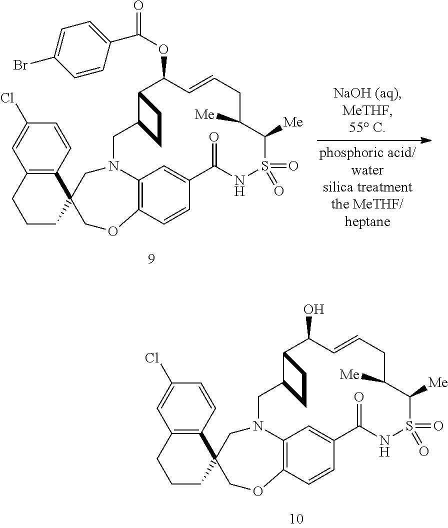

(1S,11′R,12′S,16′S,16a′R,18a′R,E)-6-chloro-16′-hydroxy-11′,12′-dimethyl-3,4,12′,13′,16′,16a′,17′,18′,18a′,19′-decahydro-1′H,2H,3′H,11′H-spiro[naphthalene-1,2′-[5,7]ethenocyclobuta[i][1,4]oxazepino[3,4-f][1]thia[2,7]diazacyclohexadecin]-8′(9′H)-one 10′,10′-dioxide (10). To a 2 L glass jacketed reactor was charged compound 9 (60 g, 76.7 mmol)), followed by 600 mL of 2-MeTHF (10 L/kg). The resulting mixture was stirred at 20° C. for 30 min. To the reactor was then charged 5M NaOH (92.05 mL, 6 equiv.) with stirring. The reaction was then stirred at 55° C. for 5 hours. To the reaction mixture was then charged 1200 mL 2-MeTHF (20 L/kg), followed by 276 mL (4.6 L/kg, 9 equiv.) of 2.5 M H₃PO₄ at 50° C. The resulting mixture was stirred at 50° C. for 10 minutes. The aqueous phase was removed after phase separation. To the reactor containing the organic phase was then charged 300 mL of water (5 L/kg) at 50° C. and the resulting mixture was stirred at 50° C. for 10 minutes. The aqueous phase was removed after phase separation. The water wash was repeated one more time. 100 w/w % SiliaMet-Thiol was then added, and the mixture was stirred at 20-45° C. for 18 hours. The mixture was then filtered and washed with 2-MeTHF. The batch was then concentrated under reduced pressure to 9 L/kg (540 mL) of the batch. The batch was then cooled to 45° C. and held for 1 hr to induce self-seeding. The resultant suspension was then cooled to 20° C., and 450 mL heptane (7.5 L/kg) was charged into the reactor. After addition, the suspension was stirred at 20° C. over one hour. (1S,11′R,12′S,16′S,16a′R,18a′R,E)-6-Chloro-16′-hydroxy-11′,12′-dimethyl-3,4,12′,13′,16′,16a′,17′,18′,18a′,19′-decahydro-1′H,2H,3′H,11′H-spiro[naphthalene-1,2′-[5,7]ethenocyclobuta[i][1,4]oxazepino[3,4-f][1]thia[2,7]diazacyclohexadecin]-8′(9′H)-one 10′,10′-dioxide (compound 10) was obtained as a white crystalline solid after filtration and washing with a 1/1 mixture of 2-MeTHF/heptane. ¹H NMR (600 MHz, CDCl₃) δ 8.53 (s, 1H), 7.70 (d, J=8.6 Hz, 1H), 7.17 (dd, J=8.6, 2.4 Hz, 1H), 7.09 (d, J=2.4 Hz, 1H), 7.00 (dd, J=8.1, 2.0 Hz, 1H), 6.96 (d, J=2.0 Hz, 1H), 6.94 (d, J=8.1 Hz, 1H), 5.85 (ddd, J=15.3, 8.4, 4.6 Hz, 1H), 5.72 (ddd, J=15.3, 8.1, 1.6 Hz, 1H), 4.28 (qd, J=7.2, 1.3 Hz, 1H), 4.25 (dd, J=8.1, 4.0 Hz, 1H), 4.09 (d, J=12.1 Hz, 1H), 4.07 (d, J=12.1 Hz, 1H), 3.84 (bd, J=14.8 Hz, 1H), 3.69 (d, J=14.1 Hz, 1H), 3.23 (d, J=14.1 Hz, 1H), 3.01 (dd, J=14.8, 9.6 Hz, 1H), 2.83-2.77 (m, 1H), 2.77-2.72 (m, 1H), 2.44 (qd, J=9.6, 4.0 Hz, 1H), 2.32 (quid, J=9.6, 1.6 Hz, 1H), 2.14-2.04 (m, 2H), 2.05-1.98 (m, 3H), 1.98-1.92 (m, 1H), 1.88 (bq, J=10.4 Hz, 1H), 1.85-1.75 (m, 2H), 1.66 (qui, J=9.6 Hz, 1H), 1.47 (d, J=7.2 Hz, 3H), 1.39 (bt, J=12.8 Hz, 1H), 1.04 (d, J=6.7 Hz, 3H); ¹³C NMR (151 MHz, CDCl₃) δ 166.5, 152.9, 140.9, 139.3, 138.8, 132.2, 132.1, 130.8, 129.6, 128.5, 126.7, 126.3, 120.9, 116.2, 115.2, 80.1, 73.3, 59.9, 58.2, 57.8, 43.6, 41.7, 37.1, 33.7, 33.6, 30.1, 28.3, 27.1, 19.2, 19.1, 15.3, 5.7. LRMS (ESI): Calcd. for C₃₂H₃₉ClN₂O₅S+Na: 621.2, found: 621.2.

### Example 11. Preparation of (1S,3′R,6′R,7′S,8′E,11′S,12′R)-6-chloro-7′-methoxy-11′,12′-dimethyl-3,4-dihydro-2H,15′H-spiro[naphthalene-1,22′[20]oxa[l3]thia[1,14]diazatetracyclo[14.7.2.0^{3,6}0.0^{19,24}]pentacosa[8,16,18,24]tetrae n]-15′-one 13′,13′-dioxide (A1)

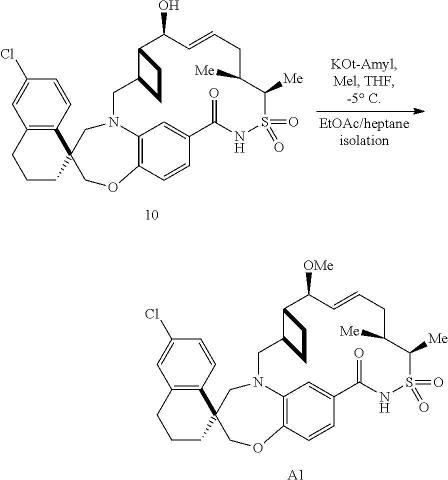

(1S,3′R,6′R,7′S,8′E,11′S,12′R)-6-chloro-7′-methoxy-11′,12′-dimethyl-3,4-dihydro-2H,15′H-spiro[naphthalene-1,22′[20]oxa[13]thia[1,14]diazatetracyclo[14.7.2.0^{3,6}.0^{19,24}]pentacosa[8,16,18,24]tetrae n]-15′-one 13′,13′-dioxide (compound A1). To a 1-L, 3-neck round bottom flask was charged (3R,7S,8E,11S,12R,22S)-6′-chloro-7-hydroxy-11,12-dimethyl-13,13-dioxo-spiro[20-oxa-13lambda6-thia-1,14-diazatetracyclo[14.7.2.03,6.0^{19,24}]pentacosa-8,16(25),17,19(24)-tetraene-22,1′-tetralin]-15-one (compound 10)(20.1 g, 33.5 mmol) followed by anhydrous tetrahydrofuran (400 mL, 20 volumes). The resulting slurry was cooled to 15° C. and then methyl iodide (5.2 mL, 83.9 mmol) was added followed by potassium tert-pentoxide (49.3 mL, 1.7 M, 83.9 mmol) as a solution in toluene at such a rate to keep the reaction temperature <20° C. After 2.5 h, the reaction was cooled to 15° C. and quenched with aqueous citric acid (80 mL, 4 volume, 1.5 M). The lower aqueous phase was removed and the upper organic phase was concentrated to 5 volumes and then diluted with ethyl acetate (300 mL, 15 volumes). The organic phase was then washed with deionized water (100 mL, 5 volumes) twice at 20° C. The organic phase was dried over sodium sulfate, polish-filtered and concentrated to 5 volumes at 65° C. The resulting solution was seeded (1 wt %) at 65° C. and aged for 30 min at 65° C. before cooling to 20° C. over 3 h. Heptane (300 mL, 15 volumes) was added dropwise over 3 h, and the mixture was then stirred overnight. The resulting solids were isolated by filtration and washed with heptane (40 mL, 2 volumes). The solids were dried in a 40° C. vacuum oven overnight. 17.01 g (82.6%) of (3R,7S,8E,11S,12R,22S)-6′-chloro-7-methoxy-11,12-dimethyl-13,13-dioxo-spiro[20-oxa-13lambda6-thia-1,14-diazatetracyclo[14.7.2.03,6.019,24]pentacosa-8,16(25),17,19(24)-tetraene-22,1′-tetralin]-15-one (A1) was isolated after drying.

All publications and patent applications cited in this specification are hereby incorporated by reference herein in their entireties and for all purposes as if each individual publication or patent application were specifically and individually indicated as being incorporated by reference and as if each reference was fully set forth in its entirety. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of synthesizing a compound of Formula BI, the compound of Formula BI having the formula 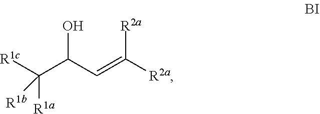
wherein the method comprises:
a) reacting a compound of Formula BII with an alkenyl boron compound and a catalyst in the presence of a base and optionally a solvent to form a product mixture comprising the compound of Formula BI, wherein the catalyst is prepared from a copper I salt or a copper II salt and a phosphine;
wherein the phosphine is at least two equivalents of a monophosphine or at least one equivalent of a diphosphine with respect to the copper I salt or is at least four equivalents of a monophosphine or at least two equivalents of a disphosphine with respect to the copper II salt;
and further wherein the sp² hybridized carbon atom of the alkenyl group that is not directly bonded to the boron atom of the alkenyl boron compound is bonded to 2 R^{2a} groups, wherein each R^{2a} is independently selected from —H, —C₁-C₆ alkyl, or a —C₆-C₁₀ aryl group, wherein the aryl group is unsubstituted or is substituted with 1 or 2 R^{3a} groups independently selected from —C₁-C₆ alkyl, —NO₂, halo, or —O—C₁-C₆; and further wherein if one of the R^{2a} groups is an aryl group, then the other R^{2a} group is not an aryl group;
wherein, BII has the formula 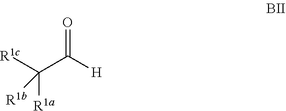
wherein
R^{1a}, R^{1b} and R^{1c} are independently selected from —H, or —C₁-C₆ alkyl, or OR^{1d} wherein R^{1d} is selected from —H, —C₁-C₆ alkyl, —Si(C₁-C₆ alkyl)₃, or C₁-C₆ alkyl-aryl, wherein the aryl in an R^{1d} group is a C₆-C₁₀ aromatic ring that is unsubstituted, or is substituted with 1, 2, or 3 substituents selected from —OH, —O—C₁-C₆ alkyl, —O—Si(C₁-C₆ alkyl)₃, halo, or C₁-C₆ haloalkyl;
or R^{1a} and R^{1b} may join to form a ring comprising 3, 4, 5, 6, 7, or 8 ring members comprising 0 or 1 oxygen atom, wherein the ring is unsubstituted or is substituted with 1 or 2 substituents selected from —OR^{1e} or —C₁-C₆—OR^{1e};
R^{1e} is selected from —H, —C₁-C₆ alkyl, —CH₂-aryl, —Si(C₁-C₆ alkyl)₃, tetrahydropyranyl, aryl, or —C═O—C₁-C₆ alkyl, wherein the aryl of an R^{1e} group is a C₆-C₁₀ aromatic ring that is unsubstituted or is substituted with 1, 2, or 3 substituents selected from —OR^{1f}, -halo, —C₁-C₆ alkyl, —C₁-C₆ haloalkyl, or —C═O—C₁-C₆ alkyl;
R^{1f} is selected from —H, —C₁-C₆ alkyl, —Si(C₁-C₆ alkyl)₃, tetrahydropyranyl, or —(C₁-C₆ alkyl)-aryl, wherein the aryl of an R^{1f} group is a C₆-C₁₀ aromatic ring that is unsubstituted, or is substituted with 1, 2, or 3 substituents selected from —OH, —O—C₁-C₆ alkyl, —O—Si(C₁-C₆ alkyl)₃, halo, or C₁-C₆ haloalkyl; and
b) reacting the product mixture with an oxidizing agent to oxidize the phosphine moieties in the phosphine to phosphine oxides to produce an oxidized phosphine.

2. The method of claim 1, wherein the method further comprises: separating crystals of the oxidized phosphine from the reaction mixture.

3. The method of claim 1 wherein the oxidizing agent is selected from H₂O₂, HOF, Ru(III)/O₂, or NaOCl.

4. The method of claim 3, wherein the oxidizing agent is an aqueous solution of H₂O₂.

5. The method of claim 2, wherein the method further comprises: reacting the separated oxidized phosphine oxide with a reducing agent to provide the phosphine.

6. The method of claim 5, wherein the reducing agent is selected from HSiCl₃, HSiCl₃:N(C₁-C₆ alkyl)₃, Si₂Cl₆, PhSiH₃, Ph₂SiH₂, Me₃SiH, Et₃SiH, PhMe₂SiH, Ph₃SiH, (Me₃Si)₃Si—H, PhCH₂SiH₃, naphthylsilane, bis(naphthyl)silane, bis(4-methylphenyl)silane, bis(fluorenyl)silane, HSi(OEt)₃, HSi(OEt)₃ with Ti(C₁-C₆ alkoxide)₄, 1,3-diphenyldisiloxane, hexamethyldisilane, TfOSi(H)(CH₃)₂, (CH₃)₂Si(H)—O—Si(CH₃)₂(H) with Cu(OTf)₂, tetramethyldisiloxane, polymethylhydrosiloxane, dialkylphosphite with I₂ and P(OPh)₃, AlH₃ with diisobutylaluminum hydride, or borane reducing agents, wherein Tf is triflate.

7. The method of claim 6, wherein the reducing agent is HSiCl₃.

8. The method of claim 1, wherein R^{1a} and R^{1b} join to form a substituted or unsubstituted ring with 3, 4, 5, or 6 ring members each of which is a carbon atom.

9. The method of claim 8, wherein R^{1a} and R^{1b} join to form a substituted or unsubstituted ring with 4 ring members each of which is a carbon atom.

10. The method of claim 8, wherein R^{1c} is —H.

11. The method of claim 8, wherein R^{1a} and R^{1b} join to form a 4 membered ring that is substituted with 1 —C₁-C₆—OR^{1e} substituent.

12. The method of claim 1, wherein R^{1a} and R^{1b} join to form a 4 membered ring that is substituted with 1 —CH₂—OR^{1e} substituent.

13. The method of claim 12, wherein R^{1a} and R^{1b} join to form a 4 membered ring that is substituted with 1 —CH₂—O—C═O—C₁-C₆ alkyl substituent.

14. The method of claim 13, wherein R^{1a} and R^{1b} join to form a 4 membered ring that is substituted with 1 —CH₂—O—C═O—CH₃ substituent.

15. The method of claim 1, wherein the compound of formula BI, has the formula IA 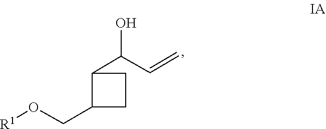
wherein R¹ is a —C═O—C₁-C₆ alkyl group.

16. The method of claim 15, wherein the compound of Formula BI has:
(i) the Formula IB 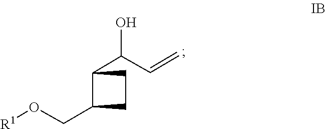
or
(ii) the Formula IC 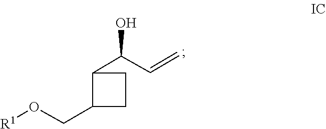
or
(iii) the Formula ID 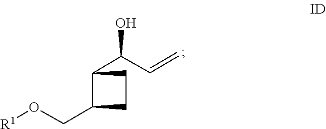
or
(iv) the Formula IE

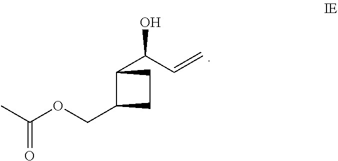

17.-19. (canceled)

20. The method of claim 15, wherein the compound of Formula BI is formed as a mixture of the compounds of Formula ID and ID′, wherein the compounds of Formula ID and ID′ have the structures:

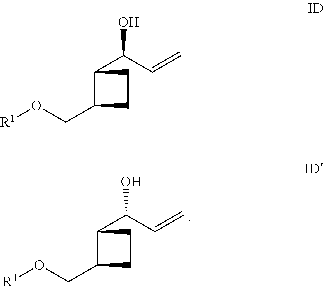

21. The method of claim 20, wherein the amount of ID to ID′ or the amount of ID′ to ID ranges from;
(i) 60:40 to 100:0; or
(ii) 65:35 to 99.9:0.1; or
(iii) 70:30 to 99.1:0.1; or
(iv) 75:25 to 99.9:0.1.

22.-24. (canceled)

25. The method of claim 20, wherein the percent of ID present in the mixture based on the total of the amount of ID and ID′ is:
(i) 60% or greater; or
(ii) 70% or greater; or
(iii) 80% or greater; or
(iv) 85% or greater; or
(v) 90% or greater; or
(vi) 95% or greater.

26.-30. (canceled)

31. The method of claim 20, wherein the compound of Formula ID has the structure IE and the compound of ID′ has the structure IE′,

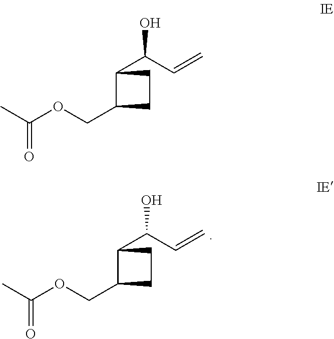

32. The method of claim 1, wherein the phosphine has at least one chiral center.

33. The method of claim 1, wherein the phosphine is a monophosphine.

34. The method of claim 1, wherein the phosphine is a diphosphine.

35. The method of claim 1, wherein the phosphine is selected from (R)-(+)-2,2′-bis(diphenylphosphino)-1,1′-binaphthyl ((R)-BINAP), 4(R)-(4,4′-bi-1,3-benzodioxole)-5,5′-diyl]bis[diphenylphosphine] ((R)-SEGPHOS), 1,1′-ferrocenediyl-bis(diphenylphosphine) (dppf), 1,3-bis(diphenylphosphino)propane (dppp), 1,2-bis(diphenylphosphino)ethane (dppe), PPh₃, 2,2′-bis(di-p-tolylphosphino)-1,1′-binaphthyl (ToIBINAP), 2,2′-bis[di(3,5-xylyl)phosphino]-1.1′-binaphthyl (XylBINAP), 5,5′-bis[di(3,5-xylyl)phosphino]-4,4′-bi-1,3-benzodioxole (DM-SEGPHOS), or (R)-1,13-bis(diphenylphosphino)-7,8-dihydro-6H-dibenzo[f,h][1,5]dioxonin ((R)-C3-TunePhos).

36. The method of claim 1, wherein the phosphine is (R)-DTBM-SEGPHOS having the structure 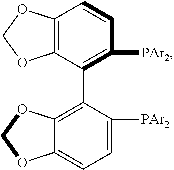
wherein Ar has the structure 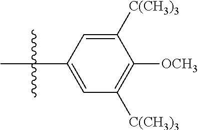 [SMILES: *c1cc(C(C)(C)C)c(OC)c(C(C)(C)C)c1]
wherein the [symbol] indicates the point of attachment to the rest of the molecule.

37. The method of claim 1, wherein the copper I salt or the copper II salt is selected from copper(I) hexafluorophosphate, copper(I) tetrafluoroborate, CuF(PPh₃)₃, CuF₂, CuF, CuI, Cu(OTf)₂, or Cu(OTf), wherein Tf is triflate.

38. The method of claim 1, wherein the catalyst is formed from a copper I salt and the copper I salt is copper(I) hexafluorophosphate or copper(I) tetrafluoroborate.

39. The method of claim 1, wherein the alkenyl boron compound is selected from 4,4,5,5-tetramethyl-2-vinyl-1,3,2-dioxaborolane. vinylBF₃K, 4,4,6-trimethyl-2-vinyl-1,3,2-dioxaborinane, vinylB(OH)₂, vinylboronic anhydride, vinylboronic acid MIDA ester, (E)-4,4,5,5-tetramethyl-2-styryl-1,3,2-dioxaborolane, (E)-4,4,5,5-tetramethyl-2-(prop-1-en-1-yl)-1,3,2-dioxaborolane, (E)-2-(3,3-dimethylbut-1-en-1-yl)-4,4,5,5-tetramethyl-1,3,2-dioxaborolane, or (E)-4,4,5,5-tetramethyl-2-(oct-1-en-1-yl)-1,3,2-dioxaborolane.

40. The method of claim 1, wherein the alkenyl boron compound is

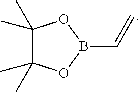

41. The method of claim 1, wherein the reaction is conducted in the presence of an organic solvent.

42. The method of claim 41, wherein the solvent is selected from isopropyl acetate, toluene, ethyl acetate, xylene, 2-methyltetrahydrofuran, tetrahydrofuran, cyclopentyl methyl ether, or t-butyl methyl ether.

43. The method of claim 1, wherein the reaction is conducted in a solvent and the solvent is isopropyl acetate.

44. The method of claim 1, wherein the compound of Formula BII is reacted with the alkenyl boron compound and the catalyst in the presence of the base and the base is selected from K₃PO₄, CsF, Cs₂CO₃, Na₂CO₃, K₂CO₃, NaF, KF, Na₃PO₄, or Cs₃PO₄.

45. The method of claim 1, wherein the compound of Formula BII is reacted with the alkenyl boron compound and the catalyst in the presence of the base and the base is K₃PO₄.

46. The method of claim 1, wherein the compound of Formula BII is reacted with the alkenyl boron compound and the catalyst at a temperature ranging from 15° C. to 50° C.; or at a temperature ranging from 20° C. to 40° C.

47. (canceled)

48. A method of synthesizing a compound of Formula IA′, the compound of Formula IA′ having the formula 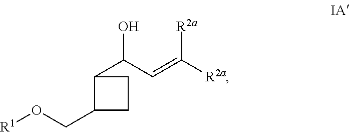
wherein the method comprises:
reacting a compound of Formula IIA with an alkenyl boron compound and a catalyst in the presence of a base and an optional solvent to form a product mixture comprising the compound of Formula IA′, wherein the catalyst is prepared from a copper I salt or a copper II salt and a phosphine, wherein the phosphine is at least two equivalents of a monophosphine or at least one equivalent of a diphosphine with respect to the copper I salt or is at least four equivalents of a monophosphine or at least two equivalents of a disphosphine with respect to the copper II salt,
and further wherein the sp² hybridized carbon atom of the alkenyl group that is not directly bonded to the boron atom of the alkenyl boron compound is bonded to 2 R^{2a} groups, wherein each R^{2a} is independently selected from —H, —C₁-C₆ alkyl, or a —C₆-C₁₀ aryl group, wherein the aryl group is unsubstituted or is substituted with 1 or 2 R^{3a} groups independently selected from —C₁-C₆ alkyl, —NO₂, halo, or —O—C₁-C₆ alkyl; and further wherein if one of the R^{2a} groups is an aryl group, then the other R^{2a} group is not an aryl group;
wherein the compound of Formula IIA has the structure 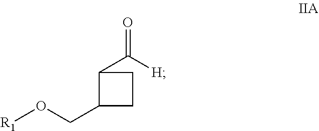
wherein R¹ is selected from —H, —C₁-C₆ alkyl, —C═O—C₁-C₆ alkyl, —C═O-aryl, —Si(C₁-C₆ alkyl)₃, tetrahydropyranyl, or —C₁-C₆ alkyl-aryl, wherein the aryl group in an R¹ group is a C₆-C₁₀ aromatic ring that is unsubstituted, or is substituted with 1, 2, or 3 substituents selected from —OH, NO₂, —O—C₁-C₆ alkyl, halo, or C₁-C₆ haloalkyl.

49. The method of claim 48, wherein the compound of Formula IA′ has:
(i) the Formula IA 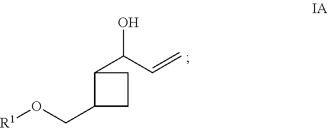
or
(ii) the Formula IB 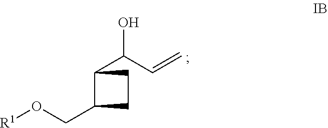
or
(iii) the Formula IC 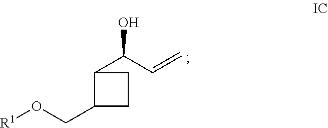
or
(iv) the Formula ID 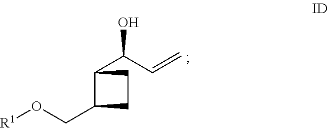
or
(v) the Formula IE

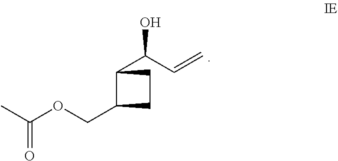

50.-53. (canceled)

54. The method of claim 49, wherein the compound of Formula IA′ is formed as a mixture of the compounds of Formula ID and ID′, wherein the compounds of Formula ID and Formula ID′ have the structures:

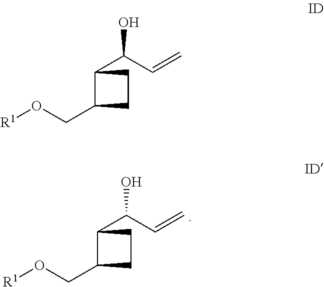

55. The method of claim 54, wherein the amount of ID to ID′ or the amount of ID′ to ID ranges from:
(i) 60:40 to 100:0; or
(ii) 65:35 to 99.9:0.1; or
(iii) 70:30 to 99.1:0.1; or
(iv) 75:25 to 99.9:0.1.

56.-58. (canceled)

59. The method of claim 54, wherein the percent of ID present in the mixture based on the total of the amount of ID and ID′ is:
(i) 60% or greater; or
(ii) 70% or greater; or
(iii) 80% or greater; or
(iv) 85% or greater; or
(v) 90% or greater; or
(vi) 95% or greater.

60.-64. (canceled)

65. The method of claim 54, wherein the compound of Formula ID has the structure IE and the compound of ID′ has the structure IE′,

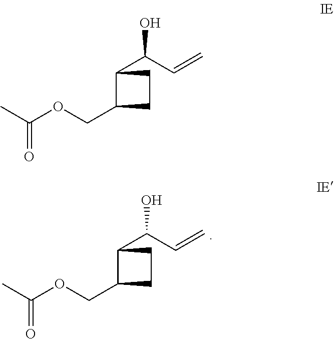

66. The method of claim 48, wherein the phosphine has at least one chiral center.

67. The method of claim 48, wherein the phosphine is a monophosphine.

68. The method of claim 48, wherein the phosphine is a diphosphine.

69. The method of claim 48, wherein the phosphine is selected from (R)-(+)-2,2′-bis(diphenylphosphino)-1,1′-binaphthyl ((R)-BINAP), 4(R)-(4,4′-bi-1,3-benzodioxole)-5,5′-diyl]bis[diphenylphosphine] ((R)-SEGPHOS), 1,1′-ferrocenediyl-bis(diphenylphosphine) (dppf), 1,3-bis(diphenylphosphino)propane (dppp), 1,2-bis(diphenylphosphino)ethane (dppe), PPh₃, 2,2′-bis(di-p-tolylphosphino)-1,1′-binaphthyl (ToIBINAP), 2,2′-bis[d (3,5-xylyl)phosphino]-1,1-binaphthyl (XylBINAP), 5,5′-bis[di(3,5-xylyl)phosphino]-4,4′-bi-1,3-benzodioxole (DM-SEGPHOS), or (R)-1,13-bis(diphenylphosphino)-7,8-dihydro-6H-dibenzo[f,h][1,5]dioxonin ((R)-C3-TunePhos).

70. The method of claim 48, wherein the phosphine is (R)-DTBM-SEGPHOS having the structure 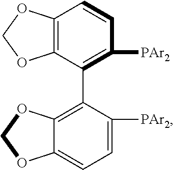
wherein Ar has the structure 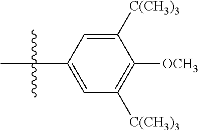 [SMILES: *c1cc(C(C)(C)C)c(OC)c(C(C)(C)C)c1]
wherein the [symbol] indicates the point of attachment to the rest of the molecule.

71. The method of claim 48, wherein the copper I salt or copper II salt is selected from copper(I) hexafluorophosphate, copper(I) tetrafluoroborate, CuF(PPh₃)₃, CuF₂, CuF, CuI, Cu(OTf)₂, or Cu(OTf), wherein Tf is triflate.

72. The method of claim 48, wherein the catalyst is formed from a copper I salt and the copper I salt is copper(I) hexafluorophosphate or copper(I) tetrafluoroforate.

73. The method of claim 48, wherein the alkenyl boron compound is selected from 4,4,5,5-tetramethyl-2-vinyl-1,3,2-dioxaborolane. vinylBF₃K, 4,4,6-trimethyl-2-vinyl-1,3,2-dioxaborinane, vinylB(OH)₂, vinylboronic anhydride, vinylboronic acid MIDA ester, (E)-4,4,5,5-tetramethyl-2-styryl-1,3,2-dioxaborolane, (E)-4,4,5,5-tetramethyl-2-(prop-1-en-1-yl)-1,3,2-dioxaborolane, (E)-2-(3,3-dimethylbut-1-en-1-yl)-4,4,5,5-tetramethyl-1,3,2-dioxaborolane, or (E)-4,4,5,5-tetramethyl-2-(oct-1-en-1-yl)-1,3,2-dioxaborolane.

74. The method of claim 48, wherein the alkenyl boron compound is

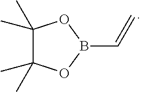

75. The method of claim 48, wherein the compound of Formula IIA is reacted with the alkenyl boron compound and the catalyst in the presence of the base and the solvent, wherein the solvent is selected from isopropyl acetate, toluene, ethyl acetate, xylene, 2-methyltetrahydrofuran, tetrahydrofuran, cyclopentyl methyl ether, or t-butyl methyl ether.

76. The method of claim 48, wherein the compound of Formula IIA is reacted with the alkenyl boron compound and the catalyst in the presence of the base and the solvent, wherein the solvent is isopropyl acetate.

77. The method of claim 48, wherein the base is selected from K₃PO₄, CsF, Cs₂CO₃, Na₂CO₃, K₂CO₃, NaF, KF, Na₃PO₄, or Cs₃PO₄.

78. The method of any claim 48, wherein the base is K₃PO₄.

79. The method of claim 48, wherein the compound of Formula BII is reacted with the alkenyl boron compound and the catalyst at a temperature ranging from 15° C. to 50° C.; or at a temperature ranging from 20° C. to 40° C.

80. (canceled)

81. The method of claim 48, wherein the method further comprises: reacting the product mixture with an oxidizing agent to oxidize the phosphine moieties in the phosphine to phosphine oxides to produce an oxidized phosphine.

82. The method of claim 81, wherein the method further comprises separating crystals of the oxidized phosphine from the reaction mixture.

83. The method of claim 81 wherein the oxidizing agent is selected from H₂O₂, HOF, Ru(III)/O₂, or NaOCl.

84. The method of claim 81, wherein the oxidizing agent is an aqueous solution of H₂O₂.

85. The method of claim 82, wherein the method further comprises: reacting the separated oxidized phosphine with a reducing agent to provide the phosphine.

86. The method of claim 85, wherein the reducing agent is selected from selected from HSiCl₃, HSiCl₃:N(C₁-C₆ alkyl)₃, Si₂Cl₆, PhSiH₃, Ph₂SiH₂, PhCH₂SiH₃, Me₃SiH, Et₃SiH, PhMe₂SiH, Ph₃SiH, (Me₃Si)₃Si—H, naphthylsilane, bis(naphthyl)silane, bis(4-methylphenyl)silane, bis(fluorenyl)silane, HSi(OEt)₃, HSi(OEt)₃ with Ti(C₁-C₆ alkoxide)₄, 1,3-diphenyldisiloxane, hexamethyldisilane, TfOSi(H)(CH₃)₂, (CH₃)₂Si(H)—O—Si(CH₃)₂(H) with Cu(OTf)₂, tetramethyldisiloxane, polymethylhydrosiloxane, dialkylphosphite with I₂ and P(OPh)₃, AlH₃ with diisobutylaluminum hydride, or borane reducing agents, wherein Tf is triflate.

87. The method of claim 86, wherein the reducing agent is HSiCl₃.

88. A compound of Formula IA having the formula 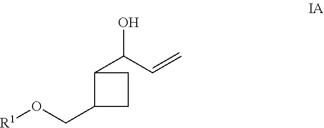
wherein R¹ is selected from —H, —C₁-C₆ alkyl, C═O—C₁-C₆ alkyl, —Si(C₁-C₆ alkyl)₃, tetrahydropyranyl, —C₁-C₆ alkyl-aryl, wherein the aryl in an R¹ group is a C₆-C₁₀ aromatic ring that is unsubstituted, or is substituted with 1, 2, or 3 substituents selected from —OH, —O—C₁-C₆ alkyl, halo, or C₁-C₆ haloalkyl.

89. The compound of claim 88, wherein the compound of Formula IA has:
(i) the Formula IB 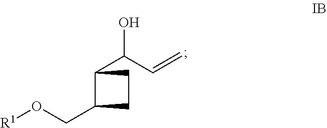
or
(ii) the Formula IC 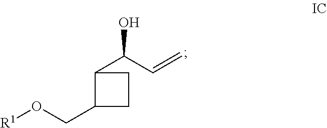
or
(iii) the Formula ID 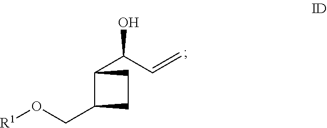
or
(iv) the Formula IE

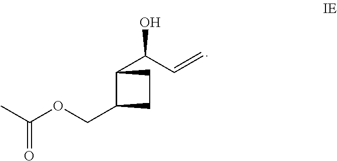

90.-92. (canceled)

93. The method of claim 15, further comprising synthesizing compound A3 using compound IA, wherein the compound A3 has the following structure:

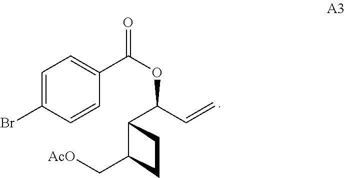

94. The method of claim 15, further comprising synthesizing compound A1 or a salt or solvate thereof using compound IA, wherein the compound A1 has the following structure:

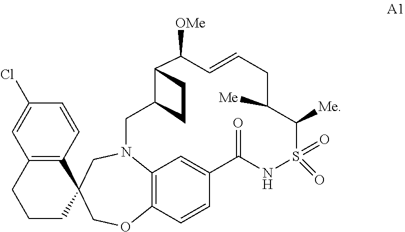

95. The method of claim 15, further comprising synthesizing compound A2 or a salt or solvate thereof using compound IA, wherein the compound A2 has the following structure:

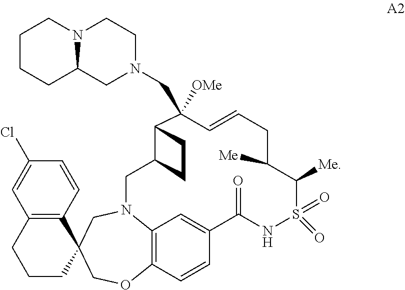

96. The method of claim 48, further comprising synthesizing compound A3 using compound IA, wherein the compound A3 has the following structure:

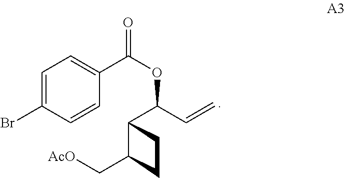

97. The method of claim 48, further comprising synthesizing compound A1 or a salt or solvate thereof using compound IA, wherein the compound A1 has the following structure:

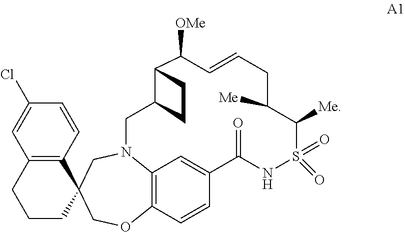

98. The method of claim 48, further comprising synthesizing compound A2 or a salt or solvate thereof using compound IA, wherein the compound A2 has the following structure: [chemical structure]
